(19) United States
(12) Patent Application Publication
(10) Pub. No.: US 2025/0228862 A1
(43) Pub. Date: Jul. 17, 2025

(54) METHODS OF USING ATR INHIBITORS

(71) Applicant: Repare Therapeutics Inc., St-Laurent (CA)

(72) Inventors: Theo GOULLET DE RUGY, Montreal (CA); Stephen MORRIS, Beaconsfield (CA); Jordan YOUNG, Saint-Lazare (CA); Michal ZIMMERMANN, Pointe-Claire (CA); Dominik GLODZIK, Boston, MA (US)

(21) Appl. No.: 18/854,217

(22) PCT Filed: Apr. 6, 2023
(86) PCT No.: PCT/CA2023/050478

§ 371 Date: Oct. 4, 2024

Related U.S. Application Data
(60) Provisional application No. 63/328,493, filed on Apr. 7, 2022.
(60) Provisional application No. 63/328,499, filed on Apr. 7, 2022.

(51) Int. Cl. A61K 31/5377; A61K 31/497; A61K 31/519; A61K 31/541; A61K 31/553; A61P 35/00

(52) U.S. Cl. CPC A61K 31/5377; A61K 31/497; A61K 31/519; A61K 31/541; A61K 31/553; A61P 35/00

(57) ABSTRACT

Disclosed are methods of treating a cancer in a subject using an ATR inhibitor wherein the cancer has been previously identified as a cancer having a loss of function of STAG2, SETD2, CDK12, ATRIP, REV3L, RAD17, CHTF8, FZR1, RAD51B, RAD51C, RAD51D, PALB2, RNASEH2A, or RNASEH2B. Also disclosed are methods of inducing cell death in an aberrant cancer cell having a loss of function of STAG2, SETD2, CDK12, ATRIP, REV3L, RAD17, CHTF8, FZR1, RAD51B, RAD51C, RAD51D, PALB2, RNASEH2A, or RNASEH2B by contacting the cell with an effective amount of an ATR inhibitor.

### FIELD OF THE INVENTION

The invention relates to the use of Ataxia-telangiectasia and RAD-3-related protein (ATR) kinase inhibitors in the treatment of cancers that harbor loss-of-function mutations in stromal antigen 2 (STAG2), SET domain containing 2 (SETD2), cyclin-dependent kinase 12 (CDK12), ATR interacting protein (ATRIP), reversionless 3-like (REV3L), RAD17, chromosome transmission fidelity factor 8 (CHTF8), fizzy and cell division cycle 20 related 1 (FZR1), RAD51B, RAD51C, RAD51D, partner and localizer of BRCA2 (PALB2), ribonuclease H2 subunit A (RNASEH2A), or ribonuclease H2 subunit B (RNASEH2B).

### BACKGROUND

DNA damage occurs continually in cells as a result of environmental insults including ultraviolet radiation, X-rays and endogenous stress factors, such as reactive oxygen and hydrolysis of bases. Cancer cells are subject to a higher rate of DNA damage inherently induced by higher rates of DNA replication in these cells. Several DNA damage response (DDR) pathways have evolved in a highly coordinated manner to help repair DNA damage and to act as a cellular checkpoint to stop the replication of cells with damaged DNA, allowing for repair functions to occur before the damaged DNA is passed on to daughter cells. Each of the identified DNA repair pathways sense and repair distinct but overlapping types of DNA damage.

One major DDR protein that acts as a key cell cycle checkpoint is the ataxia telangiectasia mutated and rad3-related (ATR) kinase, related to the family of phosphoinositide 3-kinase-related protein kinases (PIKKs). ATR is activated by single stranded (ss) DNA lesions caused by stalled replication forks or during nucleotide excision repair but is also activated by double strand breaks following DNA end resection during homologous recombination. ATR is recruited to sites of DNA damage by binding to the RPA protein that coats ssDNA along with an accessory factor called ATR-interacting protein (ATRIP). The ATR/ATRIP complex is then activated by recruitment of additional factors in the 9-1-1 complex (RAD 9, RAD1, and HUS1) which subsequently recruits the TOPBP1 protein and represents critical steps for activation of the downstream phosphorylation cascade that results in cell cycle arrest. The primary target for ATR kinase is CHK1, which when phosphorylated, targets both cdc25 proteins and Wee1 resulting in inhibition of cyclin-dependent kinase activity and cell cycle arrest in S-phase or in G2/M.

ATR has been identified as an important cancer target since it is essential for dividing cells. ATR deficient mice are embryonic lethal, however, adult mice with conditional ATR knocked out are viable with effects on rapidly proliferating tissues and stem cell populations. Cancer cells that have high levels of replication stress due to oncogenic mutations, dysfunctional G1/S checkpoint control (e.g., loss of p53 function), defects in other DNA repair pathways (e.g., ATM) or that are subject to the effects of DNA damaging agents, e.g., radiation therapy or chemotherapeutic agents, are therefore more dependent on ATR for DNA repair and survival.

Cancer cells with specific genetic mutations have unexpectedly been found to have increased sensitivity to ATR inhibitors, and patients with such mutations have an increased likelihood of achieving a beneficial response when treated with an ATR inhibitor at doses that provide a therapeutic window.

### SUMMARY OF THE INVENTION

In general, the invention provides a method of inhibiting ATR in cells having loss of function mutations in STAG2, SETD2, CDK12, ATRIP, REV3L, RAD17, CHTF8, FZR1, RAD51B, RAD51C, RAD51D, PALB2, RNASEH2A, or RNASEH2B. The method may include the step of contacting the cell with the compound disclosed herein.

In one aspect, the invention provides a treating a cancer in a subject, the method comprising administering to the subject in need thereof a therapeutically effective amount of an ATR inhibitor, wherein the cancer has been previously identified as a cancer having a loss of function (e.g., a biallelic loss of function mutation) of STAG2, SETD2, CDK12, ATRIP, REV3L, RAD17, CHTF8, FZR1, RAD51B, RAD51C, RAD51D, PALB2, RNASEH2A, or RNASEH2B.

In another aspect, the invention provides a method of treating a cancer in a subject, the method comprising administering to the subject in need thereof a therapeutically effective amount of an ATR inhibitor, wherein the cancer has a loss of function (e.g., a biallelic loss of function mutation) of STAG2, SETD2, CDK12, ATRIP, REV3L, RAD17, CHTF8, FZR1, RAD51B, RAD51C, RAD51D, PALB2, RNASEH2A, or RNASEH2B. In yet another aspect, the invention provides a method of treating a cancer in a subject, the method including the steps of:
(i) identifying the cancer as having a loss of function of STAG2, SETD2, CDK12, ATRIP, REV3L, RAD17, CHTF8, FZR1, RAD51B, RAD51C, RAD51D, PALB2, RNASEH2A, or RNASEH2B; and
(ii) administering to the subject in need thereof a therapeutically effective amount of an ATR inhibitor.

In yet another aspect, the invention provides a method of inducing cell death in an aberrant cancer cell having a loss of function of STAG2, SETD2, CDK12, ATRIP, REV3L, RAD17, CHTF8, FZR1, RAD51B, RAD51C, RAD51D, PALB2, RNASEH2A, or RNASEH2B, the method comprising contacting the cell with an effective amount of an ATR inhibitor, the effective amounts being sufficient to induce cell death in the aberrant cancer cell.

In another aspect, the invention also provides an ATR inhibitor for use in a method of treating a cancer in a subject, the method comprising administering to the subject in need thereof a therapeutically effective amount of an ATR inhibitor, wherein the cancer has been previously identified as a cancer having a loss of function (e.g., a bilallelic loss of function) of STAG2, SETD2, CDK12, ATRIP, REV3L, RAD17, CHTF8, FZR1, RAD51B, RAD51C, RAD51D, PALB2, RNASEH2A, or RNASEH2B.

In another aspect, the invention also provides an ATR inhibitor for use in a method of treating a cancer in a subject, the method comprising administering to the subject in need thereof a therapeutically effective amount of an ATR inhibitor, wherein the cancer has a loss of function (e.g., a bilallelic loss of function) of STAG2, SETD2, CDK12, ATRIP, REV3L, RAD17, CHTF8, FZR1, RAD51B, RAD51C, RAD51D, PALB2, RNASEH2A, or RNASEH2B.

In another aspect, the invention also provides an ATR inhibitor for use in a method of treating a cancer in a subject, the method including the steps of (i) identifying the cancer as having a loss of function (e.g., a bilallelic loss of function) of STAG2, SETD2, CDK12, ATRIP, REV3L, RAD17, CHTF8, FZR1, RAD51B, RAD51C, RAD51D, PALB2, RNASEH2A, or RNASEH2B; and (ii) administering to the subject in need thereof a therapeutically effective amount of an ATR inhibitor.

In another aspect, the invention also provides an ATR inhibitor for use in a method of inducing cell death in an aberrant cancer cell having a loss of function (e.g., a bilallelic loss of function) of STAG2, SETD2, CDK12, ATRIP, REV3L, RAD17, CHTF8, FZR1, RAD51B, RAD51C, RAD51D, PALB2, RNASEH2A, or RNASEH2B, the method comprising contacting the cell with an effective amount of an ATR inhibitor, the effective amounts being sufficient to induce cell death in the aberrant cancer cell.

In some embodiments of any of the above aspect, the loss of function is a loss of function of STAG2. In some embodiments, the loss of function is a loss of function of SETD2. In some embodiments, the loss of function is a loss of function of CDK12. In some embodiments, the loss of function is a loss of function of ATRIP. In some embodiments, the loss of function is a loss of function of REV3L. In some embodiments, the loss of function is a loss of function of RAD17. In some embodiments, the loss of function is a loss of function of CHTF8. In some embodiments, the loss of function is a loss of function of FZR1. In some embodiments, the loss of function is a loss of function of RAD51B. In some embodiments, the loss of function is a loss of function of RAD51C. In some embodiments, the loss of function is a loss of function of RAD51D. In some embodiments, the loss of function is a loss of function of PALB2. In some embodiments, the loss of function is a loss of function of RNASEH2A. In some embodiments, the loss of function is a loss of function of RNASEH2B.

In some embodiments, the method further includes identifying the cancer as having a biallelic STAG2, SETD2, CDK12, ATRIP, REV3L, RAD17, CHTF8, FZR1, RAD51B, RAD51C,

RAD51D, PALB2, RNASEH2A, or RNASEH2B loss of function mutation prior to the administering step or contacting step.

In some embodiments, the identifying step includes the step of:
from read counts (e.g., obtained from next-generation sequencing) for a plurality of single nucleotide variants (SNVs) including homozygous and heterozygous SNVs obtained from sequencing a sample including the cancer cell and from reference read counts, determining an integer total copy number of a locus segment within a STAG2, SETD2, CDK12, ATRIP, REV3L, RAD17, CHTF8, FZR1, RAD51B, RAD51C, RAD51D, PALB2, RNASEH2A, or RNASEH2B gene region in a cancer cell from the subject or in the cancer cell and/or two integer allele-specific copy numbers of the locus segment,
where the cancer is identified as having a STAG2, SETD2, CDK12, ATRIP, REV3L, RAD17, CHTF8, FZR1, RAD51B, RAD51C, RAD51D, PALB2, RNASEH2A, or RNASEH2B biallelic loss of function mutation,
if at least one of the integer total copy numbers and the integer allele-specific copy numbers is 0, provided that the remaining STAG2, SETD2, CDK12, ATRIP, REV3L, RAD17, CHTF8, FZR1, RAD51B, RAD51C, RAD51D, PALB2, RNASEH2A, or RNASEH2B allele, if present, includes an inactivating mutation, or
if none of the integer allele-specific copy numbers is 0 and STAG2, SETD2, CDK12, ATRIP, REV3L, RAD17, CHTF8, FZR1, RAD51B, RAD51C, RAD51D, PALB2, RNASEH2A, or RNASEH2B alleles are present, each of the alleles independently has an inactivating mutation.

In some embodiments, the determining step includes the steps of:
from the read counts and the reference read counts, determining total copy number log-ratios, allelic copy number log-odds ratios, and target coverage values for the SNVs;
segmenting the total copy number log-ratios and the allelic copy number log-odds ratios;
estimating sample purity and sample ploidy for the cancer cell from the total copy number log-ratios and the target coverage values; and
from the target coverage values, the sample purity, the sample ploidy, the total copy number log-ratios, and the allelic copy number log-odds ratios, generating an integer total copy number of a segment including a plurality of SNVs within a STAG2, SETD2, CDK12, ATRIP, REV3L, RAD17, CHTF8, FZR1, RAD51B, RAD51C, RAD51D, PALB2, RNASEH2A, or RNASEH2B gene region in the cancer cell and two integer allele-specific copy numbers of the segment.

In some embodiments, the method further includes the step of adjusting the ratios for location shift.

In some embodiments, the plurality of SNVs includes consistently covered SNVs (e.g., each of the consistently covered SNVs has the mean coverage of at least 200× reads across panel of normal samples). In some embodiments, each of the consistently covered SNVs has the mean coverage of at least 300× reads across panel of normal samples. In some embodiments, the plurality of SNVs comprises frequent SNVs, the frequent SNVs having an allele frequency of 33% to 66% in humans. In some embodiments, the plurality of SNVs includes SNVs proximal to the frequent SNVs (e.g., disposed within 300 contiguous nucleobases downstream from the frequent SNV). In some embodiments, the plurality of SNVs includes SNVs, each of the SNVs having a 5′-flanking sequence of at least 20 contiguous nucleobases including 25-75% GC content, where the 5′-flanking sequence is unique and does not include other SNVs. In some embodiments, the plurality of SNVs includes at least 20 heterozygous SNVs. In some embodiments, the reference read counts are from a panel of normal samples. In some embodiments, the plurality of SNVs includes scaffold SNVs (e.g., scaffold SNVs may be useful to limit the solution space for the integer total copy number and integer allele-specific copy numbers). In some embodiments, the gene region includes flanking regions up to 10 kilobases each. In some embodiments, the gene region includes flanking regions up to 5 kilobases each. In some embodiments, the gene region includes flanking regions up to 2 kilobases each. In some embodiments, the gene region is an exome region. In some embodiments, the gene region is a transcriptome region. In some embodiments, the gene region is a genome region. In some embodiments, the biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation includes at least one somatic mutation. In some embodiments, the biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation includes at least one germline mutation. In some embodiments, the biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation includes one somatic loss of function mutation and one germline loss of function mutation.

In some embodiments, the ATR inhibitor is a compound of formula (I):

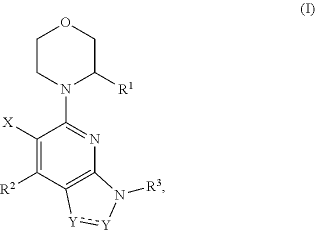

or a pharmaceutically acceptable salt thereof,
wherein
[symbol] is a double bond, and each Y is independently N or CR⁴; or [symbol] is a single bond, and each Y is independently NR^{Y}, carbonyl, or C(R^{Y})₂; wherein each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

In some embodiments, the ATR inhibitor is a compound of formula (II):

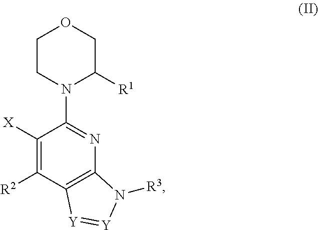

or a pharmaceutically acceptable salt thereof,
wherein
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or—SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

In some embodiments, the ATR inhibitor is selected from the group consisting of compounds 43, 57, 62, 87, 93, 94, 95, 99, 100, 106, 107, 108, 109, 111, 112, 113, 114, 115, 116, 118, 119, 120, 121, 122, 123, 135, 147, 148, and pharmaceutically acceptable salts thereof.

In some embodiments, the ATR inhibitor is compound 43 or a pharmaceutically acceptable salt thereof. In some embodiments, the ATR inhibitor is compound 121 or a pharmaceutically acceptable salt thereof. In some embodiments, the ATR inhibitor is compound 122 or a pharmaceutically acceptable salt thereof.

In some embodiments, the route of administration is an oral administration.

In some embodiments, the method does not comprise the step of administering or contacting with a PARP inhibitor. In some embodiments, the PARP inhibitor is selected from the group consisting of talazoparib, niraparib, rucaparib, olaparib, AZD5305, veliparib, iniparib, 2X-121, CEP-9722, AZD9574, pamiparib, and pharmaceutically acceptable salts thereof. In some embodiments, the ATR inhibitor is administered as a monotherapy.

In some embodiments, the cancer is renal cell carcinoma, mature B-cell neoplasm, endometrial cancer, ovarian cancer, fallopian tube cancer, primary peritoneal cancer, colorectal cancer, skin cancer, small bowel cancer, non-small cell lung cancer, melanoma, bladder cancer, pancreatic cancer, head and neck cancer, mesothelioma, glioma, prostate cancer, breast cancer, or esophagogastric cancer.

In some embodiments, the loss of function is a loss of function of STAG2. In some embodiments, the cancer is renal cell carcinoma, endometrial cancer, acute myeloid leukemia, bladder cancer, uterine cancer, or stomach cancer.

In some embodiments, the loss of function is a loss of function of SETD2. In some embodiments, the cancer is mesothelioma, kidney renal clear cell carcinoma, kidney renal papillary cell carcinoma, lung adenocarcinoma, cholangiocarcinoma, uveal melanoma, bladder urothelial carcinoma, cervical squamous cell carcinoma, endocervical adenocarcinoma, breast invasive carcinoma, lung squamous cell carcinoma, esophageal carcinoma, head and neck squamous cell carcinoma, or ovarian serous cystadenocarcinoma.

In some embodiments, the loss of function is a loss of function of CDK12. In some embodiments, the cancer is prostate adenocarcinoma, ovarian serous cystadenocarcinoma, uterine corpus endometrial carcinoma, skin cutaneous melanoma, lung adenocarcinoma, or breast invasive carcinoma.

In some embodiments, the loss of function is a loss of function of ATRIP. In some embodiments, the cancer is uveal melanoma, cervical squamous cell carcinoma, endocervical adenocarcinoma, stomach adenocarcinoma, head and neck squamous cell carcinoma, or kidney renal clear cell carcinoma.

In some embodiments, the loss of function is a loss of function of REV3L. In some embodiments, the cancer is uterine corpus endometrial carcinoma, stomach adenocarcinoma, prostate adenocarcinoma, lung squamous cell carcinoma, cervical squamous cell carcinoma, endocervical adenocarcinoma, skin cutaneous melanoma, or lung adenocarcinoma.

In some embodiments, the loss of function is a loss of function of RAD17. In some embodiments, the cancer is stomach adenocarcinoma, prostate adenocarcinoma, or breast invasive carcinoma.

In some embodiments, the loss of function is a loss of function of CHTF8. In some embodiments, the cancer is prostate adenocarcinoma.

In some embodiments, the loss of function is a loss of function of FZR1. In some embodiments, the cancer is head and neck squamous cell carcinoma, skin cutaneous melanoma, or kidney renal papillary cell carcinoma.

In some embodiments, the loss of function is a loss of function of RAD51B. In some embodiments, the cancer is uterine corpus endometrial carcinoma, kidney renal papillary cell carcinoma, cervical squamous cell carcinoma, endocervical adenocarcinoma, skin cutaneous melanoma, or breast invasive carcinoma.

In some embodiments, the loss of function is a loss of function of RAD51C. In some embodiments, the cancer is skin cutaneous melanoma, uterine corpus endometrial carcinoma, ovarian serous cystadenocarcinoma, or breast invasive carcinoma.

In some embodiments, the loss of function is a loss of function of RAD51D. In some embodiments, the cancer is ovarian serous cystadenocarcinoma, stomach adenocarcinoma, or skin cutaneous melanoma.

In some embodiments, the loss of function is a loss of function of PALB2. In some embodiments, the cancer is prostate adenocarcinoma, lung adenocarcinoma, liver hepatocellular carcinoma, breast invasive carcinoma, uterine corpus endometrial carcinoma, lung squamous cell carcinoma, ovarian serous cystadenocarcinoma, or stomach adenocarcinoma.

In some embodiments, the loss of function is a loss of function of RNASEH2A. In some embodiments, the loss of function is a loss of function of RNASEH2B. In some embodiments, the cancer is sarcoma, bladder urothelial carcinoma, chronic lymphocytic leukemia.

### Definitions

The term “aberrant,” as used herein, refers to different from normal. When used to describe enzymatic activity, aberrant refers to activity that is greater or less than a normal control or the average of normal non-diseased control samples. Aberrant activity may refer to an amount of activity that results in a disease, where returning the aberrant activity to a normal or non-disease-associated amount (e.g., by administering a compound or using a method as described herein), results in reduction of the disease or one or more disease symptoms. The aberrant activity can be measured by measuring the modification of a substrate of the enzyme in question; a difference of greater or equal to a 2-fold change in activity could be considered as aberrant. Aberrant activity could also refer to an increased dependence on a particular signaling pathway as a result of a deficiency in a separate complementary pathway.

The term “acyl,” as used herein, represents a group-C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C₁₋₇ alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl) oyl.” These groups represent a carbonyl group substituted by aryl, cycloalkyl, or heterocyclyl, respectively. Each of “aryloyl,” “cycloalkanoyl,” and “(heterocyclyl) oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkoxy,” as used herein, represents a chemical substituent of formula-OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula-L-O—R, where L is C₁₋₆ alkylene, and R is C₁₋₆ alkyl. An optionally substituted alkoxyalkyl is an alkoxyalkyl that is optionally substituted as described herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; aryl; aryloxy; azido; cycloalkyl; cycloalkoxy; cycloalkenyl; cycloalkynyl; halo; heterocyclyl; (heterocyclyl)oxy; heteroaryl; hydroxy; nitro; thiol; silyl; cyano; alkylsulfonyl; alkylsulfinyl; alkylsulfenyl; ═O; ═S; —SO₂R, where R is amino or cycloalkyl; =NR′, where R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula-N(R^{N1})₂ or—NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)2-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “amino,” as used herein, represents-N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., NHR^{N1}), where R^{N1} is independently —OH, SO₂ORN₂, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, refers to a divalent aryl group. An optionally substituted arylene is an arylene that is optionally substituted as described herein for aryl.

The term “aryloxy,” as used herein, represents a chemical substituent of formula-OR, where R is an aryl group, unless otherwise specified. In optionally substituted aryloxy, the aryl group is optionally substituted as described herein for aryl.

The term “ATM,” as used herein, represents ATM serine/threonine kinase.

The term “ATR inhibitor,” as used herein, represents a compound that upon contacting the enzyme ATR kinase, whether in vitro, in cell culture, or in an animal, reduces the activity of ATR kinase, such that the measured ATR kinase IC₅₀ is 10 μM or less (e.g., 5 μM or less or 1 μM or less). For certain ATR inhibitors, the ATR kinase IC₅₀ may be 100 nM or less (e.g., 10 nM or less, or 1 nM or less) and could be as low as 100 pM or 10 pM. Preferably, the ATR kinase IC₅₀ is 0.1 nM to 1 μM (e.g., 0.1 nM to 750 nM, 0.1 nM to 500 nM, or 0.1 nM to 250 nM).

The term “ATR kinase,” as used herein, refers to Ataxia-telangiectasia and RAD-3-related protein kinase.

The term “azido,” as used herein, represents an —N₃ group.

The term “BRCA2,” as used herein, represents a breast cancer type 2 susceptibility gene or protein.

The term “cancer,” as used herein, refers to all types of cancer, neoplasm or malignant tumors found in mammals (e.g., humans), including leukemia, carcinomas and sarcomas. Non-limiting examples of cancers that may be treated with a compound or method provided herein include prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, ampullary cancer, colorectal cancer, and pancreatic cancer. Additional non-limiting examples may include, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, and prostate cancer.

The term “carbocyclic,” as used herein, represents an optionally substituted C3-16 monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)-group.

The term “carcinoma,” as used herein, refers to a malignant new growth made up of epithelial cells tending to infiltrate the surrounding tissues and give rise to metastases. Non-limiting examples of carcinomas that may be treated with a compound or method provided herein include, e.g., medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “cyano,” as used herein, represents-CN group.

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkoxy,” as used herein, represents a chemical substituent of formula-OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C_{3-C10} cycloalkyl), unless otherwise specified. Cycloalkyl groups may be monocyclic or bicyclic. Bicyclic cycloalkyl groups may be of bicyclo [p.q.0] alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo [p.q.r] alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q] alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.] heptyl, 2-bicyclo[2.2.1.] heptyl, 5-bicyclo[2.2.1.] heptyl, 7-bicyclo[2.2.1.] heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is amino or cycloalkyl; =NR′, where R′ is H, alkyl, aryl, or heterocyclyl; or—CON(R^{A}) 2, where each R^{A} is independently H or alkyl, or both R^{A}, together with the atom to which they are attached, combine to form heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

“Disease” or “condition” refer to a state of being or health status of a patient or subject capable of being treated with the compounds or methods provided herein.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryl alkyl,” as used herein, represents an alkyl group substituted with a heteroaryl group, each as defined herein. The heteroaryl and alkyl portions may be optionally substituted as the individual groups described herein.

The term “heteroarylene,” as used herein, represents a divalent heteroaryl. An optionally substituted heteroarylene is a heteroarylene that is optionally substituted as described herein for heteroaryl.

The term “heteroaryloxy,” as used herein, refers to a structure-OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four, five, or six substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an—OH group.

The term “isotopically enriched,” as used herein, refers to the pharmaceutically active agent with the isotopic content for one isotope at a predetermined position within a molecule that is at least 100 times greater than the natural abundance of this isotope. For example, a composition that is isotopically enriched for deuterium includes an active agent with at least one hydrogen atom position having at least 100 times greater abundance of deuterium than the natural abundance of deuterium. Preferably, an isotopic enrichment for deuterium is at least 1000 times greater than the natural abundance of deuterium. More preferably, an isotopic enrichment for deuterium is at least 4000 times greater (e.g., at least 4750 times greater, e.g., up to 5000 times greater) than the natural abundance of deuterium.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, e.g., acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “lymphoma,” as used herein, refers to a cancer arising from cells of immune origin. Non-limiting examples of T and B cell lymphomas include non-Hodgkin lymphoma and Hodgkin disease, diffuse large B-cell lymphoma, follicular lymphoma, mucosa-associated lymphatic tissue (MALT) lymphoma, small cell lymphocytic lymphoma-chronic lymphocytic leukemia, Mantle cell lymphoma, mediastinal (thymic) large B-cell lymphoma, lymphoplasmacytic lymphoma-Waldenstrom macroglobulinemia, peripheral T-cell lymphoma (PTCL), angioimmunoblastic T-cell lymphoma (AITL)/follicular T-cell lymphoma (FTCL), anaplastic large cell lymphoma (ALCL), enteropathy-associated T-cell lymphoma (EATL), adult T-cell leukaemia/lymphoma (ATLL), or extranodal NK/T-cell lymphoma, nasal type.

The term “melanoma,” as used herein, is taken to mean a tumor arising from the melanocytic system of the skin and other organs. Melanomas that may be treated with a compound or method provided herein include, e.g., acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

The term “nitro,” as used herein, represents an —NO₂ group.

The term “oxo,” as used herein, represents a divalent oxygen atom (e.g., the structure of oxo may be shown as ═O).

The term “PARP inhibitor,” as used herein, represents a compound that upon contacting PARP, whether in vitro, in cell culture, or in an animal, reduces the activity of PARP, such that the measured PARP IC₅₀ is 10 μM or less (e.g., 5 μM or less or 1 μM or less). For certain PARP inhibitors, the PARP IC₅₀ may be 100 nM or less (e.g., 10 nM or less, or 1 nM or less) and could be as low as 100 pM or 10 pM. Preferably, the PARP IC₅₀ is 0.1 nM to 1 μM (e.g., 0.5 nM to 750 nM, 1 nM to 500 nM, or 1 nM to 250 nM).

The term “PARP,” as used herein, refers to poly ADP ribose polymerase (PARP).

The term “Ph,” as used herein, represents phenyl.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl) ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl) ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl) ethoxy]methyl (SEM), tetrahydropyranyl (THP), t-butyloxycarbonyl (Boc), and benzyloxycarbonyl (Cbz).

The term “RNAse H2A,” as used herein, refers to Ribonuclease H2, subunit A.

The term “RNAse H2B,” as used herein, refers to Ribonuclease H2, subunit B.

The term “sarcoma” generally refers to a tumor which is made up of a substance like the embryonic connective tissue and is generally composed of closely packed cells embedded in a fibrillar or homogeneous substance. Non-limiting examples of sarcomas that may be treated with a compound or method provided herein include, e.g., a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “scaffold SNV,” as used herein, represent frequent, well-covered single nucleotide variants outside the target gene region and spaced throughout the chromosome carrying the target gene region.

The term “STAG2”, as used herein, refers to Stromal Antigen 2.

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone-enol, enamine-imine, amide-imidic acid, nitroso-oxime, ketene-ynol, and amino acid-ammonium carboxylate.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

The term “total copy number log-ratio,” as used herein, refers to a cancer cell over control cell signal ratio. The total copy number log-ratio deviations from an average of 0 for a given region suggest signal intensity to be higher (if greater than 0) or lower (if less than 0) than expected for two chromosomal copies. The total copy number log-ratio, also known as LogR, may be estimated using GenomeStudio® software from Illumina.

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy). A disease or condition may be a cancer. Non-limiting examples of cancers include, e.g., renal cell carcinoma, mature B-cell neoplasms, endometrial cancer, ovarian cancer, colorectal cancer, skin cancer (non-melanoma), small bowel cancer, non-small cell lung cancer, melanoma, bladder cancer, pancreatic cancer, head and neck cancer, mesothelioma, glioma, prostate cancer, breast cancer, and esophagogastric cancer.

### BRIEF DESCRIPTION OF THE DRAWINGS

FIG. 1A: Inhibition of the proliferation of STAG2-KO cells by compound 121. The STAG2 gene was knocked out in RPE1-hTERT-ATP53 cells using CRISPR-Cas9 (clone 1 and 2). Cells were then seeded in 96well plate format and treated with indicated concentrations of compound 121 for a period of 7 days. Treatment was refreshed every 4 days. Confluency was then measured using microscopy and normalized to the non-treated condition.

FIG. 1B: compound 121 induces cell death preferentially in STAG2-KO cells. RPE1-hTERT-ATP53-STAG2 KO cells were seeded in 6-well plate format and treated with the indicated dose of compound 121 for 10 days. Cells were then fixed and stained using a crystal violet-methanol solution. Colonies were counted, and percentage of survival was determined by normalizing the number of colonies after treatment to the number of colonies in the non-treated conditions.

FIG. 2: compound 121 preferentially induces cell death in bladder carcinoma cells deficient for STAG2. The STAG2 gene was knocked out in T24 cells using CRISPR-Cas9 (clone 1 and 2). Cells were seeded in 6-well plate format and treated with indicated dose of compound 121 for 9 days. Cells were then fixed and stained using a crystal violet-methanol solution. Colonies were counted, and percentage of survival was determined by normalizing the number of colonies after treatment to the number of colonies in the non-treated conditions.

FIG. 3A: Representative dose-response curves of compound 121-treated MCF10A cells after transfection with either a non-targeting siRNA (siCTRL) or siRNA targeting SETD2 (siSETD2). Cell viability was determined by a CellTiter Glo (CTG) assay. Data (open symbols) were fitted to a 4-parameter dose-response model (solid lines). Knockdown of ATM (siATM; ATM is a known determinant of compound 121 sensitivity) was used as a positive control.

FIG. 3B: compound 121 IC₅₀ values from CTG cell viability assays in MCF10A and RPE1 TP53−/− cell lines transfected with the indicated siRNAs. Data from ≥2 independent experiments (circles) with mean (bars)±SD (error bars).

FIG. 3C: Hela cells are not sensitive to compound 121 upon SETD2 knockdown. Note that in this cell line, the positive control (siATM) also does not sensitize to compound 121. Data from 3 independent experiments (circles) with mean (bars)±SD (error bars).

FIG. 4A: Representative immunoblot showing SETD2 expression in whole-cell extracts from six SETD2 wild-type (WT) renal and colorectal carcinoma cell lines and loss of SETD2 in three SETD2-mutated (LOF) cell lines. Vinculin was used as a loading control.

FIG. 4B: compound 121 IC₅₀ values from CellTiter Glo viability assays of SETD2 WT and LOF cell lines treated with compound 121 for 4-8 population doublings. Circles represent mean IC₅₀ values from ≥3 independent experiments. Bars, mean of each cohort (WT or LOF); error bars, ±SD.

FIG. 5A: Dose-response curves of isogenic CDK12 wild-type (+/+) and knockout (−/−) RPE1 TP53−/− cells treated with compound 121. Cell viability was measured by CellTiter Glo assays. Open symbols represent mean from three independent experiments±SD. Solid lines show a non-linear least square fit to a four-parameter dose-response model. Approximately a 2× shift in IC₅₀ was observed.

FIG. 5B: Dose-response curves of isogenic CDK12 wild-type (+/+) and knockout (−/−) DLD1 cells treated with compound 121. Cell viability was measured by CellTiter Glo assays. Open symbols represent mean from three independent experiments ±SD. Solid lines show a non-linear least square fit to a four-parameter dose-response model. Approximately a 3× shift in IC₅₀ was observed.

FIG. 6A: Dose-response curves of isogenic RNASEH2B wild-type (+/+) and knockout (−/−) RPE1 TP53−/− cells treated with compound 121. Cell viability was measured by CellTiter Glo assays. Open symbols represent mean from three independent experiments±SD. Solid lines show a non-linear least square fit to a four-parameter dose-response model. FIG. 6A also shows immunoblots showing RNASEH2B protein status in whole cell extracts of the indicated cell lines. α-Actinin was used as a loading control. Approximately a 4× shift in IC₅₀ was observed.

FIG. 6B: Dose-response curves of isogenic RNASEH2B wild-type (+/+) and knockout (−/−) 5637 cells treated with compound 121. Cell viability was measured by CellTiter Glo assays. Open symbols represent mean from three independent experiments±SD. Solid lines show a non-linear least square fit to a four-parameter dose-response model. FIG. 6B also shows immunoblots showing RNASEH2B protein status in whole cell extracts of the indicated cell lines. α-Actinin was used as a loading control. Approximately a 4× shift in IC₅₀ was observed.

FIG. 7A: Growth of subcutaneously grafted 5637 RNASEH2B+/+ bladder cancer cell line tumors in immunodeficient mice. Mice were treated by oral gavage with either vehicle or indicated doses of compound 121 once daily at a 2 day on/4 day off weekly schedule. Data are shown as mean±SEM. N=8 mice/group.

FIG. 7B: Growth of subcutaneously grafted 5637 RNASEH2B−/− bladder cancer cell line tumors in immunodeficient mice. Mice were treated by oral gavage with either vehicle or indicated doses of compound 121 once daily at a 2 day on/4 day off weekly schedule. Data are shown as mean±SEM. N=8 mice/group.

FIG. 8A: Growth of subcutaneously implanted triple-negative breast cancer (TNBC) PDX tumors with RNASEH2B IHC loss in immunodeficient mice. Mice were treated by oral gavage with either vehicle or indicated doses of compound 121 once daily at a 3 day on/4 day off intermittent weekly schedule. Data are shown as mean±SEM. N=9 mice/group.

FIG. 8B: Immunohistochemical (IHC) staining of the PDX model from FIG. 8A (top) and a control, RNase H2-proficient PDX (bottom) with an RNase H2-specific antibody.

FIG. 9A: compound 121 dose-response curves of RPE1 Cas9 TP53−/− cells transduced with either a control sgRNA (sgAAVS1) or two sgRNAs targeting RAD51B. Cell viability was determined with a CellTiter Glo assay. Open symbols show mean viability from three independent experiments±SD. Solid lines represent a non-linear least square fit to a four-parameter dose-response model.

FIG. 9B: compound 121 dose-response curves of RPE1 Cas9 TP53−/− cells transduced with either a control sgRNA (sgAAVS1) or two sgRNAs targeting RAD51C. Cell viability was determined with a CellTiter Glo assay. Open symbols show mean viability from three independent experiments±SD. Solid lines represent a non-linear least square fit to a four-parameter dose-response model.

FIG. 9C: compound 121 dose-response curves of RPE1 Cas9 TP53−/− cells transduced with either a control sgRNA (sgAAVS1) or two sgRNAs targeting RAD51D. Cell viability was determined with a CellTiter Glo assay. Open symbols show mean viability from three independent experiments±SD. Solid lines represent a non-linear least square fit to a four-parameter dose-response model.

FIG. 10A: Fold shift in compound 121 IC₅₀ values from CellTiter Glo cell viability assays in MCF10A cells transfected with non-targeting siRNAs (siCTRL), a positive control siRNA targeting ATM (siATM) or siRNAs targeting indicated STEP2 genes. Circles represent values from ≥2 independent experiments with mean (bars)±SD (error bars). Data are normalized to siCTRL. Values larger than 1 represent increased sensitivity as compared to siCTRL.

FIG. 10B: Fold shift in compound 121 IC₅₀ values from CellTiter Glo cell viability assays in RPE1 TP53−/− cells transfected with non-targeting siRNAs (siCTRL), a positive control siRNA targeting ATM (siATM) or siRNAs targeting indicated STEP2 genes. Circles represent values from ≥2 independent experiments with mean (bars)±SD (error bars). Data are normalized to siCTRL. Values larger than 1 represent increased sensitivity as compared to siCTRL.

FIG. 10C: Fold shift in compound 121 IC₅₀ values from CellTiter Glo cell viability assays in Hela cells transfected with non-targeting siRNAs (siCTRL), a positive control siRNA targeting ATM (siATM) or siRNAs targeting indicated STEP2 genes. Circles represent values from ≥2 independent experiments with mean (bars)±SD (error bars). Data are normalized to siCTRL. Values larger than 1 represent increased sensitivity as compared to siCTRL.

FIG. 11A: Dose-response curves of isogenic ARID1A wild-type (WT) and knockout (ARID1A−/−) HCT116 cells treated with AZD-6738. Cell growth (confluency) was measured by IncuCyte automated microscopy. Data (circles) were fitted to a four-parameter dose-response model (solid lines).

FIG. 11B: Dose-response curves of isogenic ARID1A wild-type (WT) and knockout (ARID1A−/−) MCF1A cells treated with AZD-6738. Cell growth (confluency) was measured by IncuCyte automated microscopy. Data (circles) were fitted to a four-parameter dose-response model (solid lines).

FIG. 11C: Dose-response curves of isogenic ARID1A wild-type (WT) and knockout (ARID1A−/−) BEAS2B cells treated with AZD-6738. Cell growth (confluency) was measured by IncuCyte automated microscopy. Data (circles) were fitted to a four-parameter dose-response model (solid lines).

FIG. 12: EC50 values for the ATRI AZD-6738 from IncuCyte growth assays in a panel of cancer cell lines either wild-type for ARID1A (WT) or carrying deleterious ARID1A alterations (LOF). Data (circles) from two independent experiments with mean (bars)±SD (error bars).

### DETAILED DESCRIPTION

In general, the invention relates to the use of an ATR inhibitor, or a pharmaceutically acceptable salt thereof, for the treatment of cancers or for inducing cell death in cancer cells. The cancers included herein may be, e.g., cancers having a loss of function (e.g., a biallelic loss of function) of STAG2, SETD2, CDK12, ATRIP, REV3L, RAD17, CHTF8, FZR1, RAD51B, RAD51C, RAD51D, PALB2, RNASEH2A, or RNASEH2B.

Advantageously, cancers having a loss of function (e.g., a biallelic loss of function) of STAG2, SETD2, CDK12, ATRIP, REV3L, RAD17, CHTF8, FZR1, RAD51B, RAD51C, RAD51D, PALB2, RNASEH2A, or RNASEH2B may be particularly sensitive to ATR inhibitor therapy (e.g., ATR inhibitor monotherapy). For example, cancers having a loss of function of CDK12, RNASEH2A, or RNASEH2B may be treated using the ATR inhibitor therapy, e.g., as an ATR inhibitor monotherapy.

STAG2 is a gene encoding for the SA-2 protein, key member of the cohesin complex. This complex forms a ring-shaped structure that maintains two DNA chromatid together. This physical proximity of DNA molecules is essential for different processes such as DNA replication, DNA repair and chromosome segregation during mitosis. Some cancer patients show alteration of the STAG2 gene, likely contributing to the genetic instability observed in tumors. Because of this higher level of genetic instability, STAG2 deficient tumors are likely to rely on DDR signaling for survival.

SETD2 is a gene encoding for a histone methyltransferase that specifically methylates lysine 36 of histone H3. The function of the SETD2 protein is important for many cellular processes, including transcription, DNA replication and DNA repair. Some tumors carry deleterious alterations in the SETD2 gene, which may increase the levels of replication stress and genome instability in these tumors.

The CDK12 gene encodes for a cyclin-dependent kinase required for DNA transcription. Deleterious alterations of CDK12 found in some cancers lead to reduced expression of genes involved in DNA repair and DNA replication, resulting in reduced ability of these tumors to repair damaged DNA and recover from replication stress.

The ATRIP and RAD17 genes encode proteins required for proper function of the ATR pathway. When these genes are altered in tumors, levels of replication stress may increase.

The REV3L gene encodes the catalytic subunit of DNA polymerase 5, a protein complex required by DNA repair by translesion synthesis. Deleterious alterations of REV3L in cancers are expected to diminish the tumors' ability to repair DNA damage and recover from replication stress.

The CHTF8 gene encodes a subunit of the alternative replication factor C (RFC) complex, consisting of DSCC1, CHTF8 and CHTF18. This protein complex is required for proper DNA replication, sister chromatid cohesion and DNA repair. Some tumors carry deleterious alterations of CHTF8, potentially leading to DNA repair and replication defects.

FZR1 (also known as CDH1) is a gene encoding for a regulatory subunit of the Anaphase-Promoting Complex (APC/C). This protein complex is required for proper cell division. Deleterious alterations in the FZR1 gene are expected to disrupt the tumor cells' ability to recover from replication stress and increase genome instability.

RAD51B, RAD51C, RAD51D and PALB2 are genes encoding for proteins required for DNA repair by homologous recombination (HR). Together, the protein products of these genes support proper loading of the RAD51 recombinase on DNA by the BRCA2 protein and are therefore essential for the completion of HR DNA repair. Deleterious alterations of RAD51B, RAD51C, RAD51D or PALB2 can be found in some cancers, leading to defects in HR and consequent increase in genome instability.

The RNASEH2A and RNASEH2B genes encode for subunits of the Ribonuclease H2 (RNase H2) complex, which is responsible for the removal of ribonucleotides mistakenly incorporated into DNA by DNA polymerases during DNA replication and DNA repair (aka ‘genomic ribonucleotides’). Some tumors harbor deleterious alterations in the RNASEH2A or RNASEH2B genes, leading to aberrant processing of genomic ribonucleotides and elevated levels of replication stress and genome instability.

Due to the underlying DNA repair defects and genome instability, cancers with STAG2, SETD2, CDK12, ATRIP, REV3L, RAD17, CHTF8, FZR1, RAD51B, RAD51C, RAD51D, PALB2, RNASEH2A, or RNASEH2B mutations may respond to the treatment with an ATR inhibitor at doses that allow the establishment of a therapeutic window over healthy proliferating cells.

### ATR Inhibitors

An ATR inhibitor may be a compound that upon contacting the enzyme ATR kinase, whether in vitro, in cell culture, or in an animal, reduces the activity of ATR kinase, such that the measured ATR kinase IC₅₀ is 10 μM or less (e.g., 5 μM or less or 1 μM or less). For certain ATR inhibitors, the ATR kinase IC₅₀ may be 100 nM or less (e.g., 10 nM or less, or 1 nM or less) and could be as low as 100 pM or 10 pM. Preferably, the ATR kinase IC₅₀ is 0.1 nM to 1 μM (e.g., 0.1 nM to 750 nM, 0.1 nM to 500 nM, or 0.1 nM to 250 nM).

Non-limiting examples of ATR inhibitors include, e.g.:

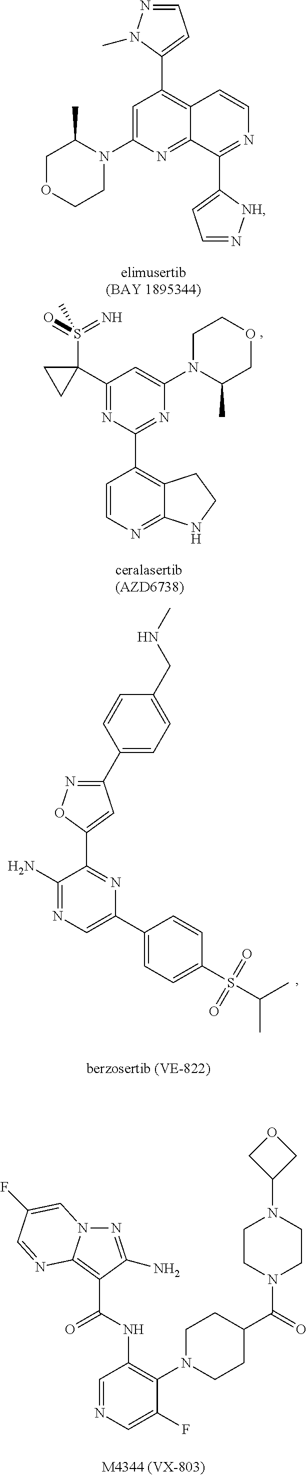 [SMILES: C[C@@H]1COCCN1c1cc(-c2ccnn2C)c2ccnc(-c3ccn[nH]3)c2n1 ; C[C@@H]1COCCN1c1cc(C2([S@](C)(=N)=O)CC2)nc(-c2ccnc3c2CCN3)n1 ; CNCc1ccc(-c2cc(-c3nc(-c4ccc(S(=O)(=O)C(C)C)cc4)cnc3N)on2)cc1 ; Nc1nn2cc(F)cnc2c1C(=O)Nc1cncc(F)c1N1CCC(C(=O)N2CCN(C3COC3)CC2)CC1]

and pharmaceutically acceptable salts thereof.

Non-limiting examples of ATR inhibitors include, e.g., those described in, e.g., International Application Publication Nos. WO 2020087170, WO 2018218197, WO 2020259601, WO 2019036641, WO 2020049017, WO2019154365, WO 2020103897, WO2021233376, WO2022028598, WO2022012484, WO2022002245, WO2022002243 each of which is incorporated by reference herein; U.S. Pat. Nos. 11,028,076, 10,745,420, 10,301,324, 10,196,405, 9,663,535, 9,549,932, 8,552,004, and 8,841,308, each of which is incorporated by reference herein; and U.S. Patent Application Publication No. 2019/0055240 and 2019/0300547, which is incorporated by reference herein.

In one embodiment, an ATR inhibitor is a compound of formula (I):

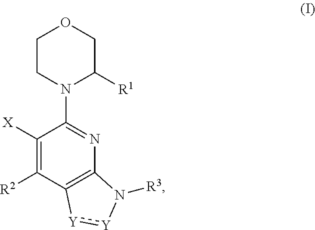

or a pharmaceutically acceptable salt thereof,
where
[symbol] is a double bond, and each Y is independently N or CR⁴; or [symbol] is a single bond, and each Y is independently NR^{Y}, carbonyl, or C(R^{Y})₂; where each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or—SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

The ATR inhibitor may be, e.g., a compound of formula (II):

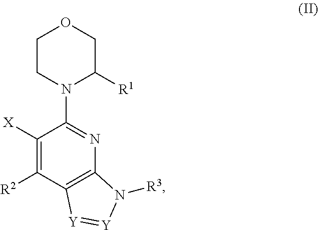

or a pharmaceutically acceptable salt thereof,
where
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

In some embodiments, in the compound of formula (II), (I), or (I-b):
each Y is independently N or CR⁴;
R¹ is H or optionally substituted C₁₋₆ alkyl;
R² is optionally substituted C₁₋₆ alkyl, optionally substituted C₃₋₈ cycloalkyl, optionally substituted C₂₋₉ heterocyclyl, optionally substituted C₆₋₁₀ aryl, optionally substituted C₁₋₉ heteroaryl, optionally substituted C₁₋₉ heteroaryl C₁₋₆ alkyl, —N(R⁵)₂, —CON(R⁶)₂, —SO₂N(R⁶)₂, or —SO₂R^{5A};
R³ is optionally substituted C₁₋₉ heteroaryl;
each R⁴ is independently H or optionally substituted C₁₋₆ alkyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}, where each R^{5A} is independently optionally substituted C₁₋₆ alkyl or optionally substituted C₃₋₈ cycloalkyl; or both R⁵, together with the atom to which they are attached, combine to form an optionally substituted C₂₋₉ heterocyclyl;
each R^{5A} is independently optionally substituted C₁₋₆ alkyl or optionally substituted C₃₋₈ cycloalkyl; and
each R⁶ is independently hydrogen, optionally substituted C₁₋₆ alkyl, optionally substituted C₆₋₁₀ aryl C₁₋₆ alkyl, optionally substituted C₆₋₁₀ aryl, or optionally substituted C₁₋₉ heteroaryl; or both R⁶, together with the atom to which they are attached, combine to form an optionally substituted C₂₋₉ heterocyclyl.

Methods of making compounds of formula (I) are described, e.g., in International Application No. PCT/US2019/051539, hereby incorporated by reference.

The ATR inhibitor may be, e.g., a compound of formula (I-a):

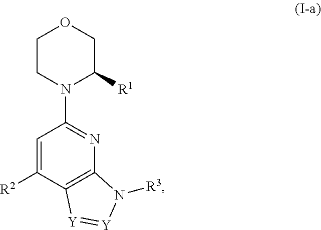

or a pharmaceutically acceptable salt thereof, where Y, R¹, R², R³, and R⁴ are as described for formula (I).

The ATR inhibitor may be, e.g., a compound of formula (I-b):

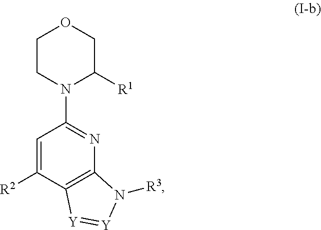

or a pharmaceutically acceptable salt thereof, where Y, R¹, R², R³, and R⁴ are as described for formula (I).

The ATR inhibitor may be, e.g., a compound of formula (IA):

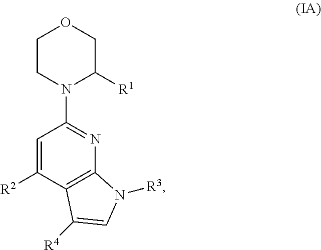

or a pharmaceutically acceptable salt thereof, where R¹, R², R³, and R⁴ are as described for formula (I).

The ATR inhibitor may be, e.g., a compound of formula (IA-a):

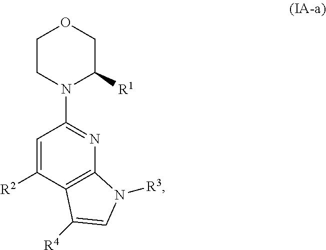

or a pharmaceutically acceptable salt thereof, where R¹, R², R³, and R⁴ are as described for formula (I).

The ATR inhibitor may be, e.g., a compound of Formula (IB):

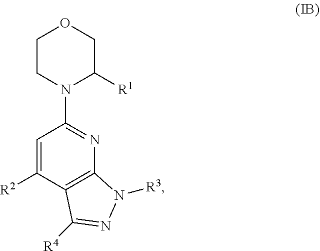

or a pharmaceutically acceptable salt thereof, where R¹, R², R³, and R⁴ are as described for formula (I).

The ATR inhibitor may be, e.g., a compound of formula (IB-a):

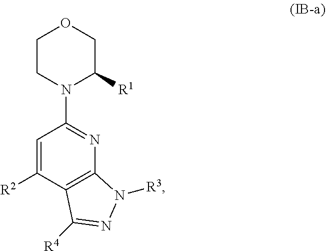

or a pharmaceutically acceptable salt thereof, where R¹, R², R³, and R⁴ are as described for formula (I).

The ATR inhibitor may be, e.g., a compound of Formula (IC):

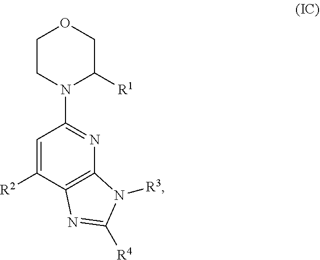

or a pharmaceutically acceptable salt thereof, where R¹, R², R³, and R⁴ are as described for formula (I).

The ATR inhibitor may be, e.g., a compound of formula (IC-a):

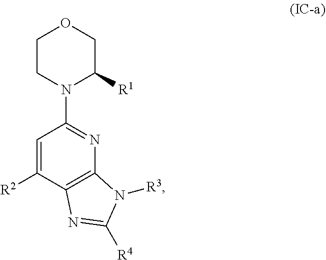

or a pharmaceutically acceptable salt thereof, where R¹, R², R³, and R⁴ are as described for formula (I).

The ATR inhibitor may be, e.g., a compound of formula (ID):

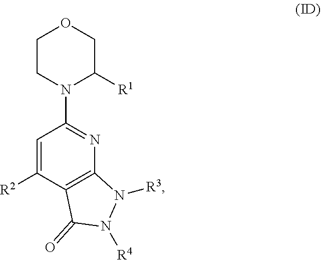

or a pharmaceutically acceptable salt thereof, where R¹, R², R³, and R⁴ are as described for formula (I).

The ATR inhibitor may be, e.g., a compound of formula (ID-a):

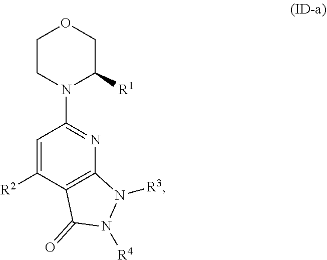

or a pharmaceutically acceptable salt thereof, where R¹, R², R³, and R⁴ are as described for formula (I).

Preferably, R¹ is methyl.

In some embodiments, R² may be, e.g., optionally substituted C₃₋₈ cycloalkyl. For example, R² may be a group of formula (A):

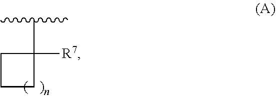

where
n is 0, 1, 2, or 3; and
R⁷ is hydrogen, alkylsulfonyl, cyano, —CON(R^{A}) 2, —SON(R^{A}) 2, optionally substituted C₁₋₉ heteroaryl, hydroxy, or alkoxy, where each R^{A} is independently H or alkyl; or both R^{A}, together with the atom to which they are attached, combine to form C₂₋₉ heterocyclyl.

In some embodiments, R² may be, e.g., optionally substituted C₁₋₆ alkyl (e.g., optionally substituted tertiary C₃₋₆ alkyl. For example, R² may be a group of formula (B):

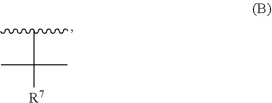

where R⁷ is hydrogen, alkylsulfonyl, cyano, —CON(R^{A}) 2, —SON(R^{A}) 2, optionally substituted C₁₋₉ heteroaryl, hydroxy, or alkoxy, where each R^{A} is independently H or alkyl; or both R^{A}, together with the atom to which they are attached, combine to form C₂₋₉ heterocyclyl.

In some embodiments, R² may be, e.g., optionally substituted non-aromatic C₂₋₉ heterocyclyl.

In some embodiments, R² may be, e.g.:

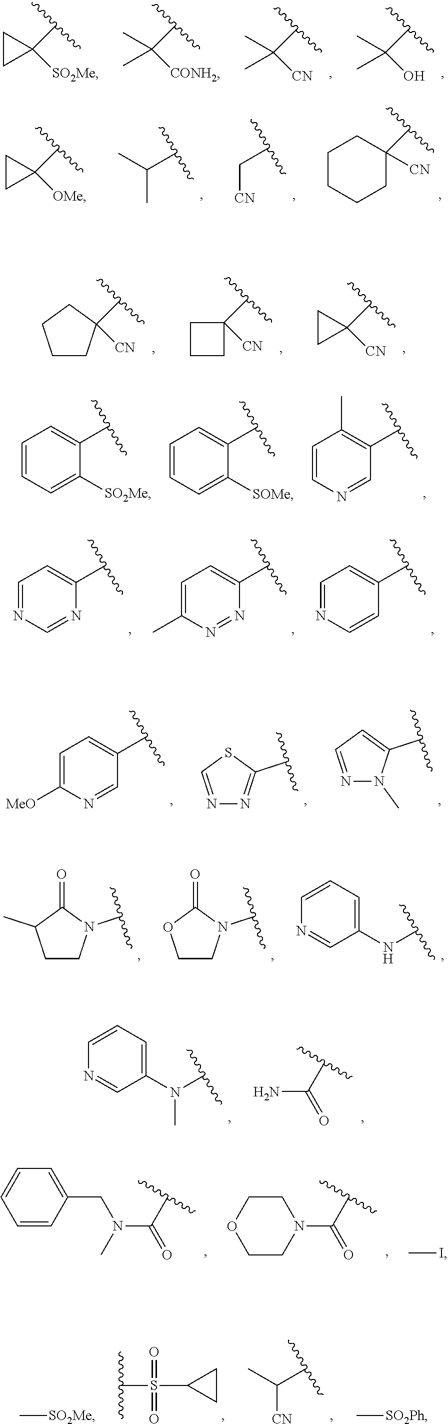

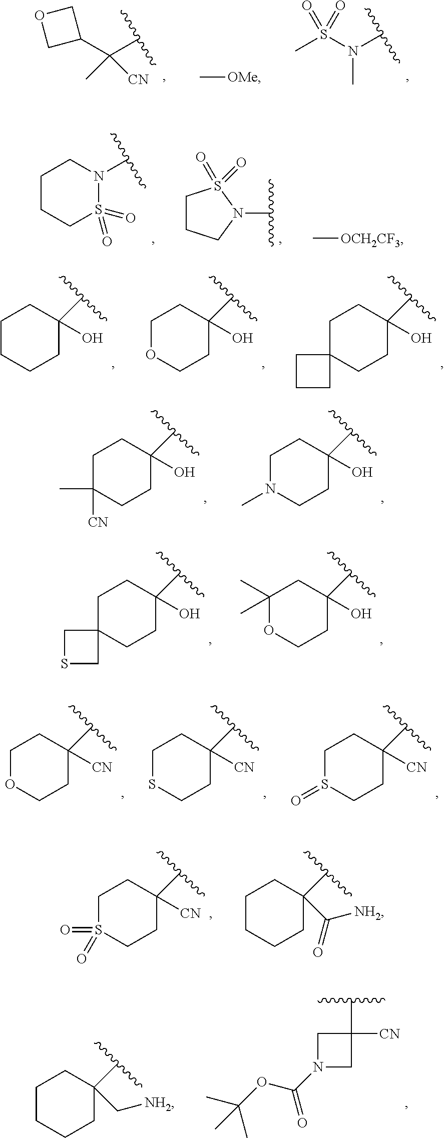

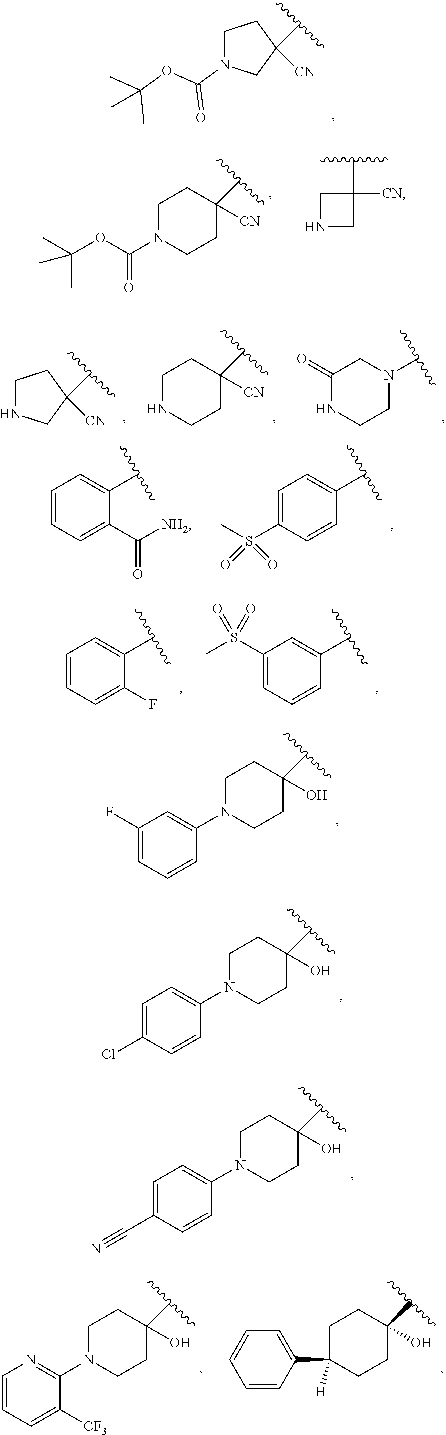

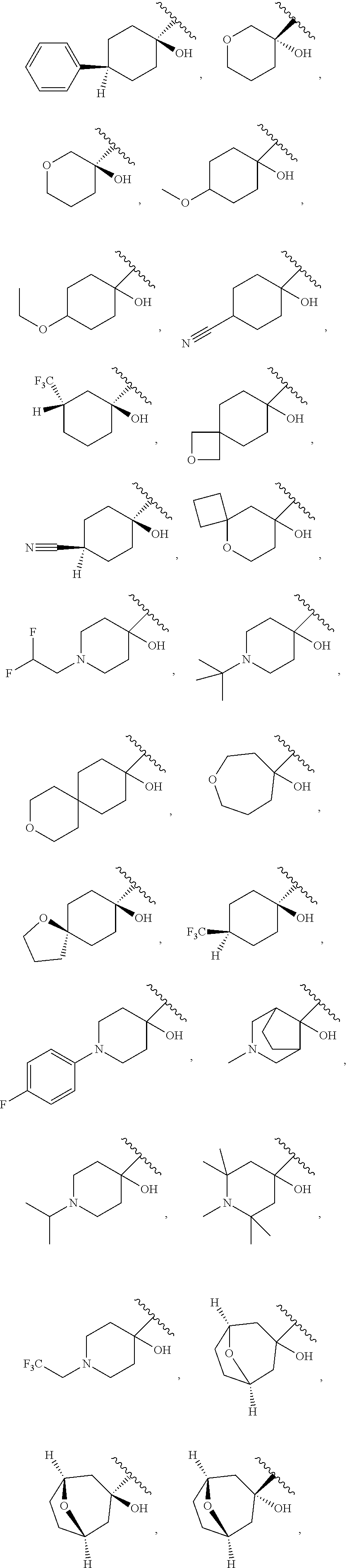

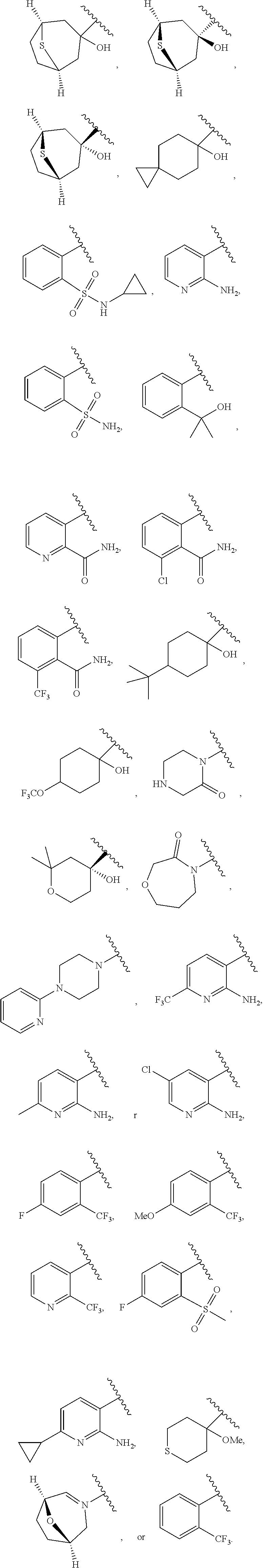

In some embodiments, R³ may be, e.g., optionally substituted, monocyclic C₁₋₉ heteroaryl including at least one nitrogen atom (e.g., two nitrogen atoms). For example, R³ may be a group of formula (C):

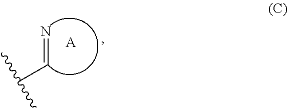

where A is optionally substituted, monocyclic C₁₋₉ heteroaryl ring.

In some embodiments, A may be, e.g., a group of formula (C1):

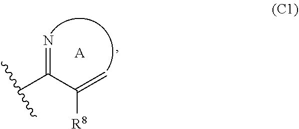

where R⁸ is hydrogen, halogen, or optionally substituted C₁₋₆ alkyl.

In some embodiments, R³ may be, e.g.,

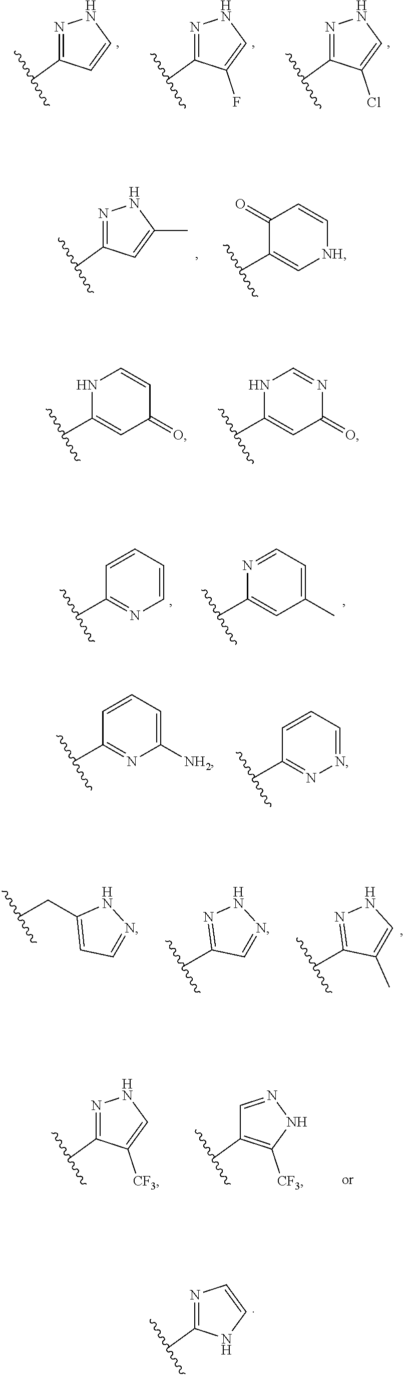

In some embodiments, R³ may be, e.g.,

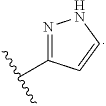 [SMILES: *c1cc[nH]n1]

In some embodiments, R⁴ may be, e.g., hydrogen.

The ATR inhibitor may be, e.g., a compound listed in Table 1 below or a pharmaceutically acceptable salt thereof.

TABLE 1
An ATR inhibitor may be isotopically enriched (e.g., enriched for deuterium).

| | |
|---|---|
| 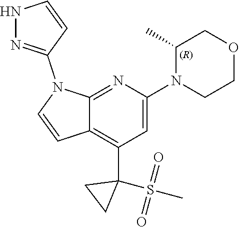 | 1 |
| 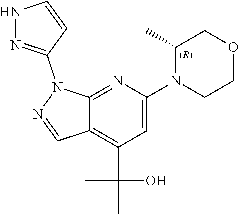 | 2 |
| 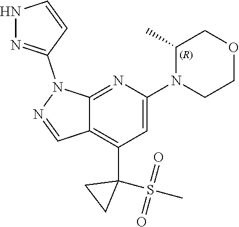 | 3 |
| 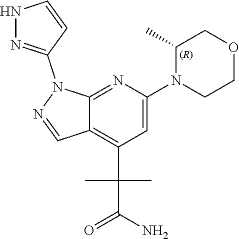 | 4 |
| 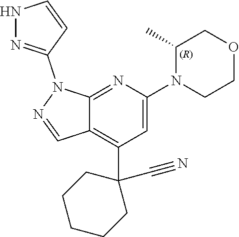 | 5 |
| 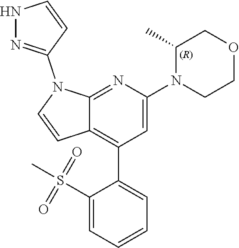 | 6 |
| 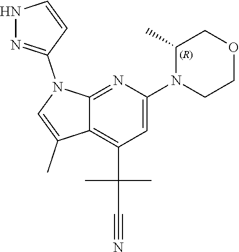 | 7 |
| 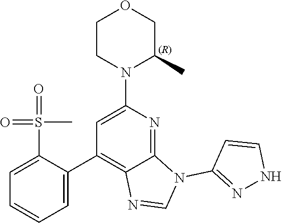 | 8 |
| 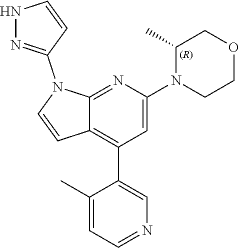 | 9 |
| 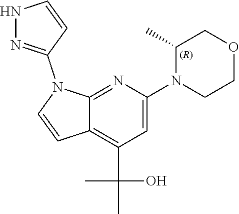 | 10 |
| 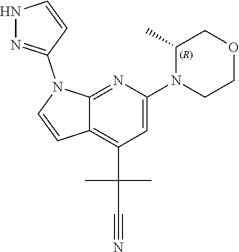 | 11 |
| 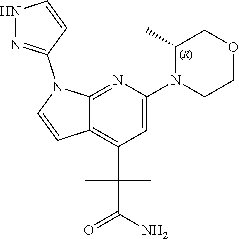 | 12 |
| 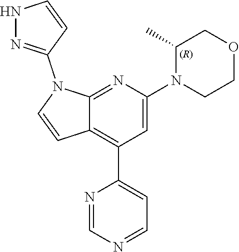 | 13 |
| 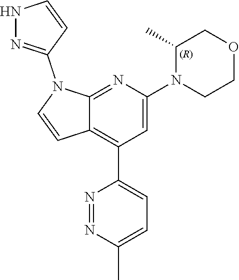 | 14 |
| 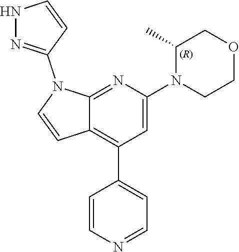 | 15 |
| 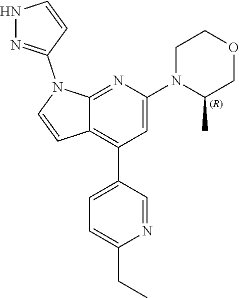 | 16 |
| 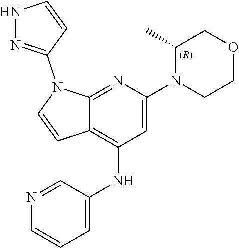 | 17 |
| 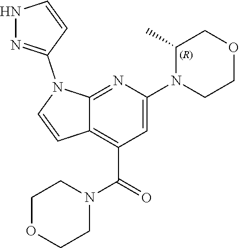 | 18 |
| 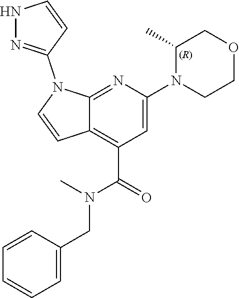 | 19 |
| 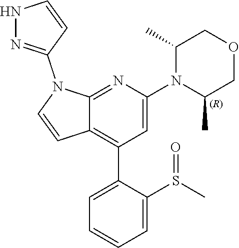 | 20 |
| 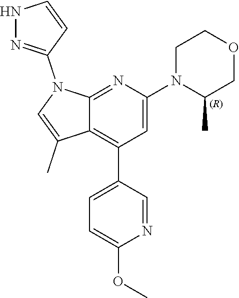 | 21 |
| 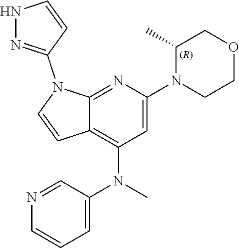 | 22 |
| 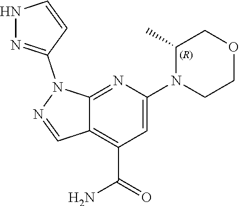 | 23 |
| 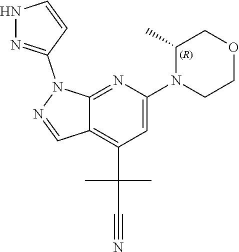 | 24 |
| 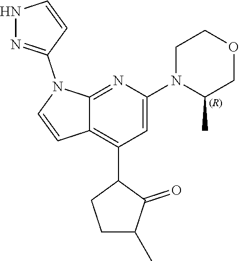 | 25 |
| 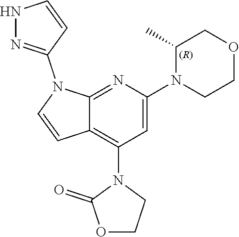 | 26 |
| 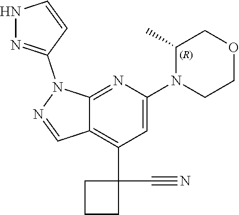 | 27 |
| 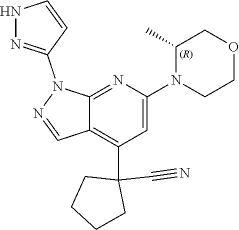 | 28 |
| 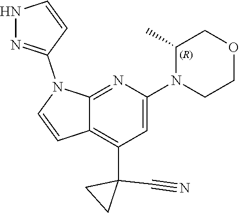 | 29 |
| 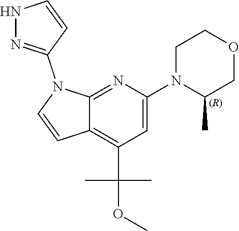 | 30 |
| 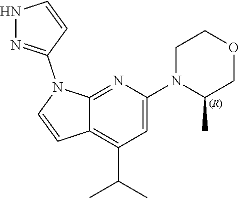 | 31 |
| 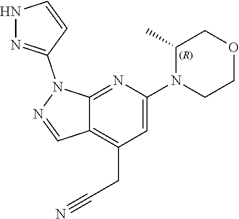 | 32 |
| 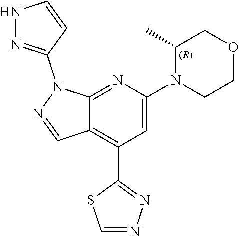 | 33 |
| 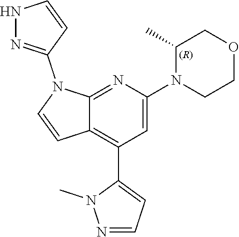 | 34 |
| 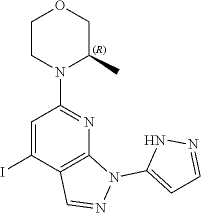 | 42 |
| 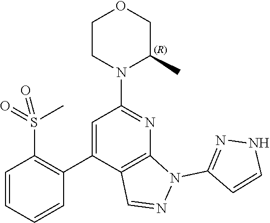 | 43 |
| 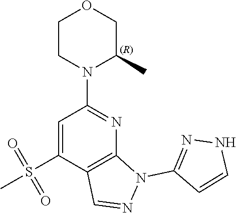 | 44 |
| 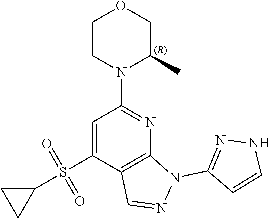 | 45 |
| 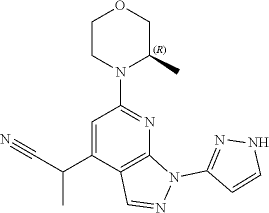 | 47 |
| 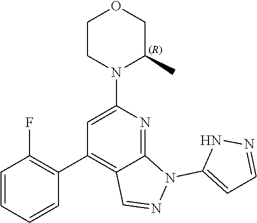 | 48 |
| 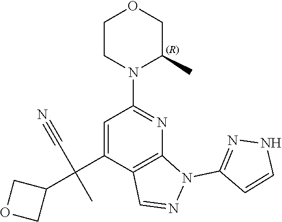 | 49 |
| 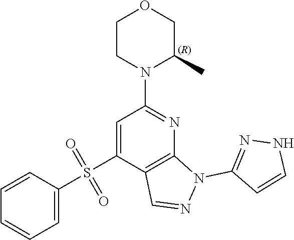 | 50 |
| 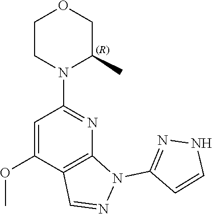 | 51 |
| 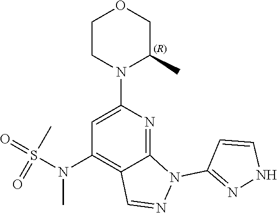 | 52 |
| 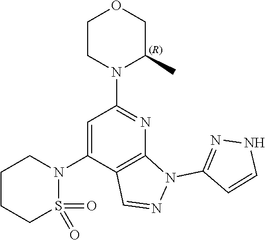 | 53 |
| 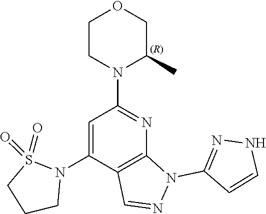 | 54 |
| 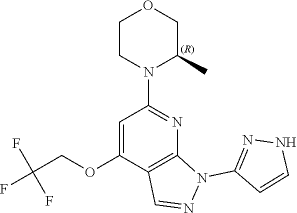 | 55 |
| 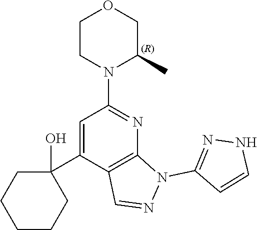 | 56 |
| 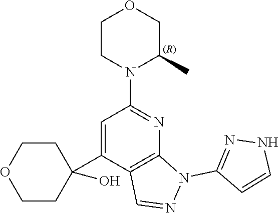 | 57 |
| 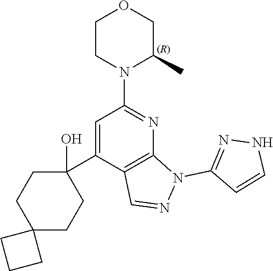 | 58 |
| 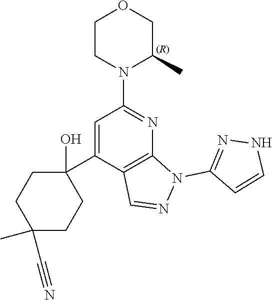 | 59 |
| 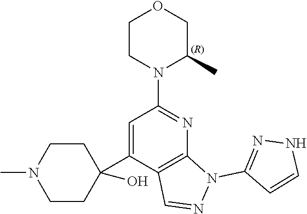 | 60 |
| 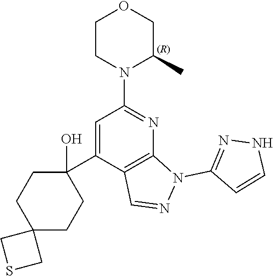 | 61 |
| 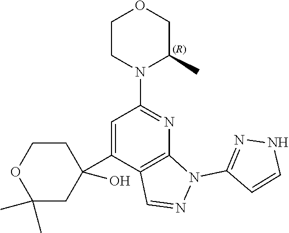 | 62 |
| 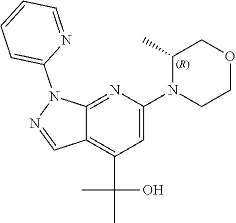 | 63 |
| 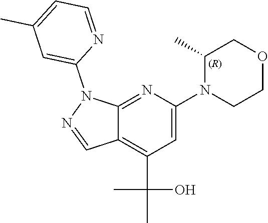 | 64 |
| 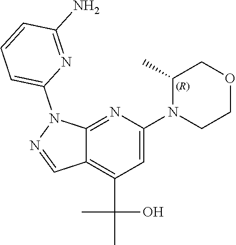 | 65 |
| 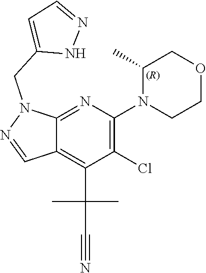 | 66 |
| 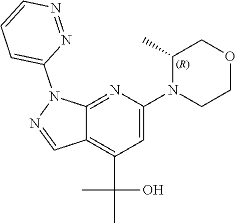 | 67 |
| 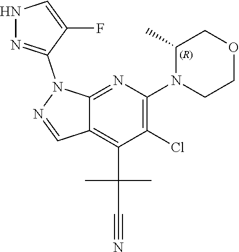 | 68 |
| 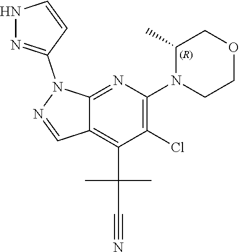 | 69 |
| 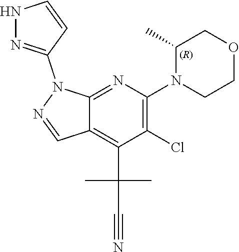 | 70 |
| 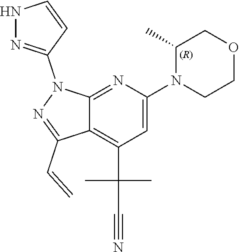 | 71 |
| 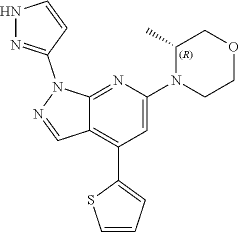 | 72 |
| 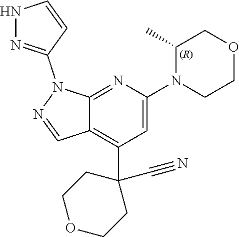 | 73 |
| 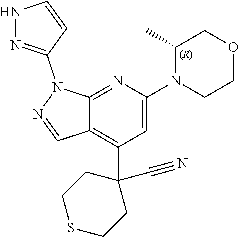 | 74 |
| 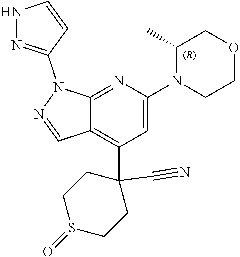 | 75 |
| 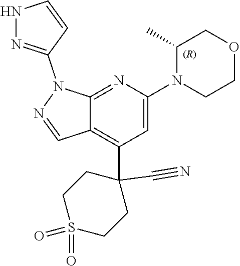 | 76 |
| 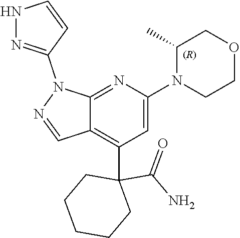 | 77 |
| 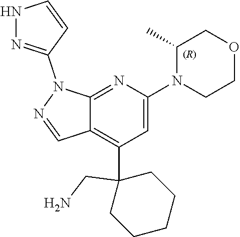 | 78 |
| 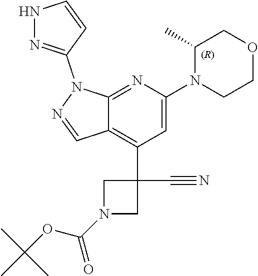 | 79 |
| 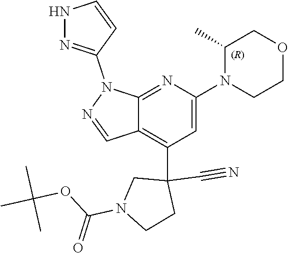 | 80 |
| 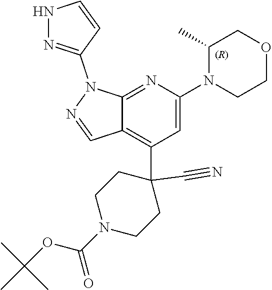 | 81 |
| 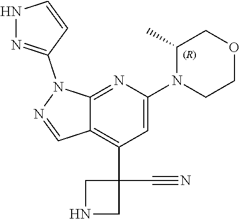 | 82 |
| 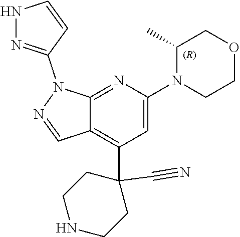 | 83 |
| 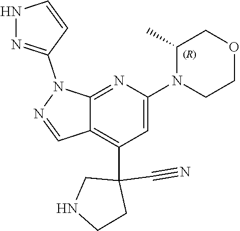 | 84 |
| 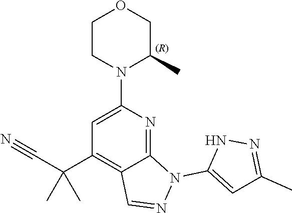 | 86 |
| 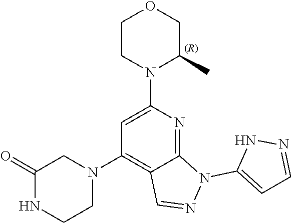 | 87 |
| 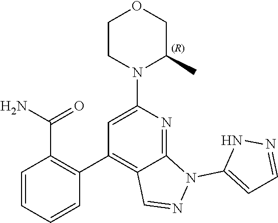 | 88 |
| 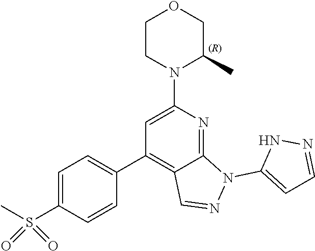 | 89 |
| 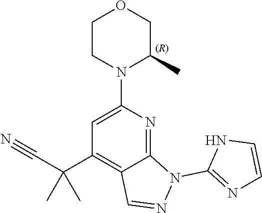 | 90 |
| 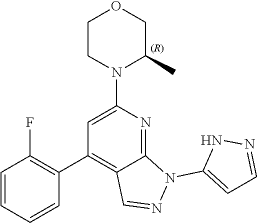 | 91 |
| 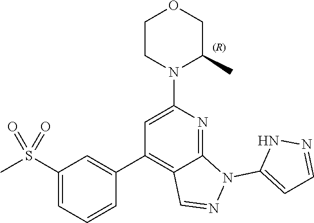 | 92 |
| 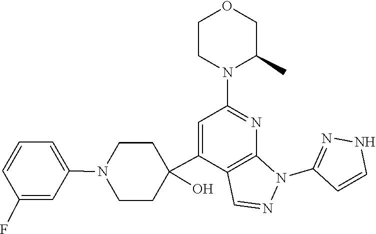 [SMILES: C[C@@H]1COCCN1c1cc(C2(O)CCN(c3cccc(F)c3)CC2)c2cnn(-c3cc[nH]n3)c2n1] | 93 |
| 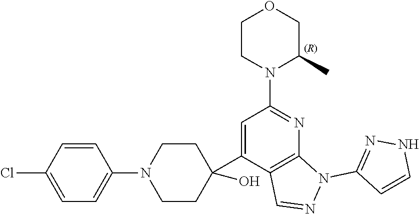 | 94 |
| 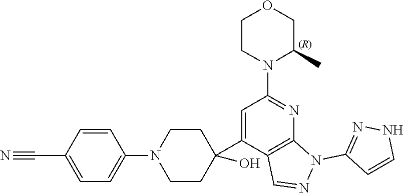 | 95 |
| 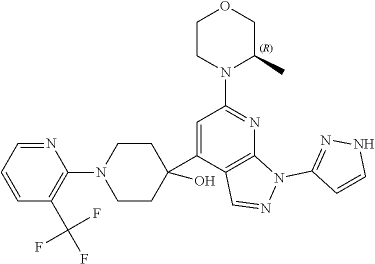 | 96 |
| 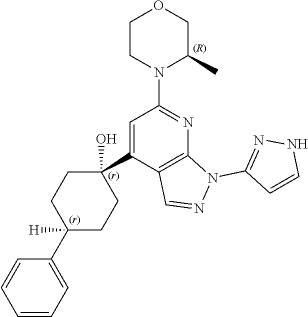 | 97 |
| 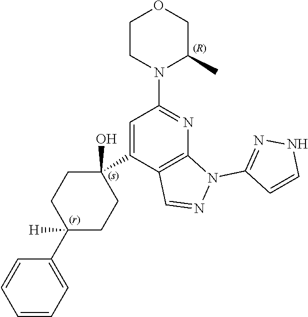 | 98 |
| 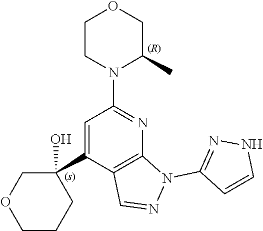 | 99 |
| 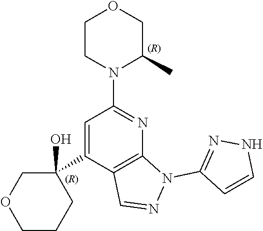 | 100 |
| 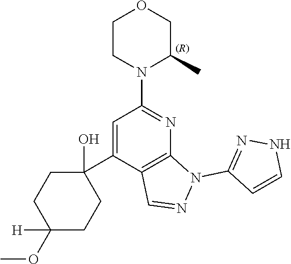 | 101 |
| 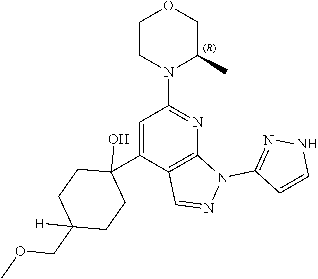 | 102 |
| 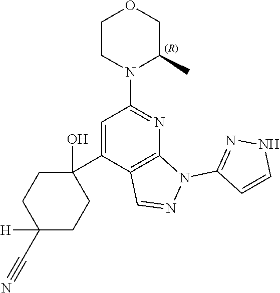 | 103 |
| 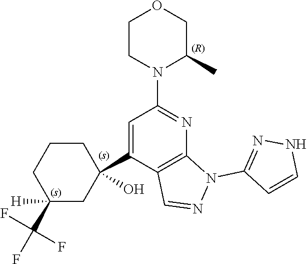 | 104 |
| 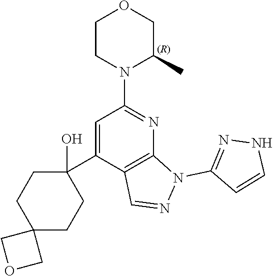 | 105 |
| 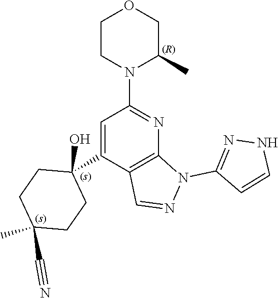 | 106 |
| 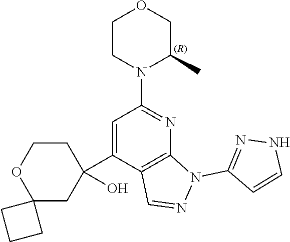 | 107 |
| 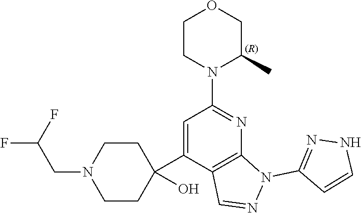 | 108 |
| 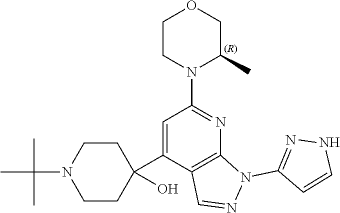 | 109 |
| 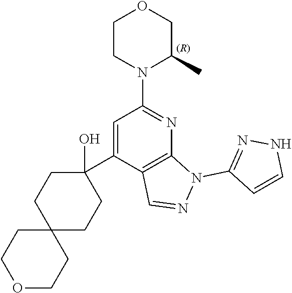 | 110 |
| 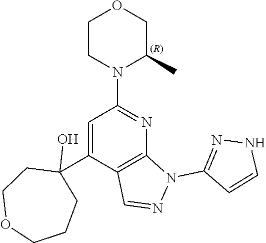 | 111 |
| 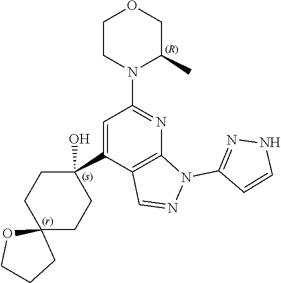 | 112 |
| 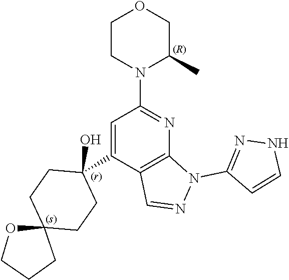 | 113 |
| 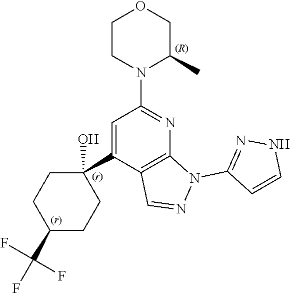 | 114 |
| 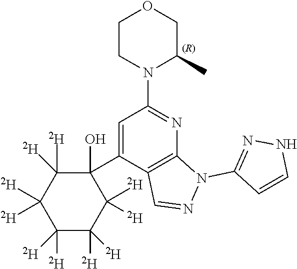 | 115 |
| 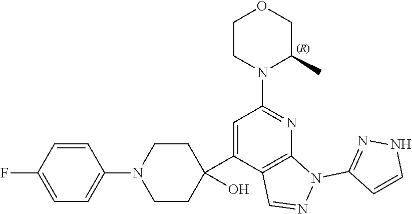 | 116 |
| 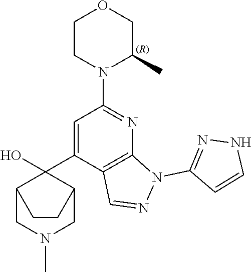 | 117 |
| 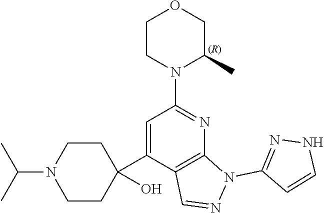 | 118 |
| 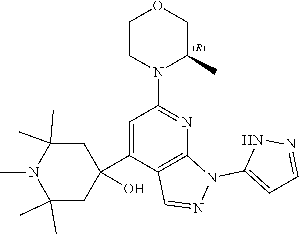 | 119 |
| 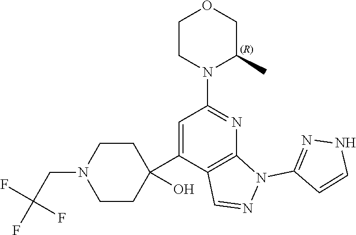 | 120 |
| 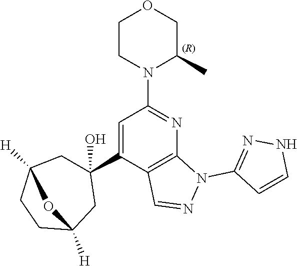 | 158 |
| 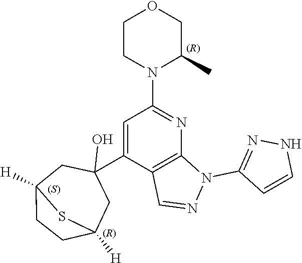 | 122 |
| 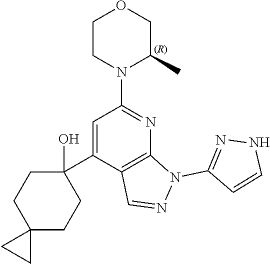 | 123 |
| 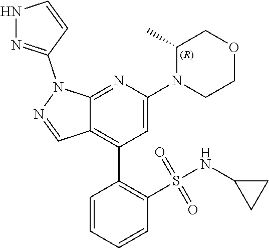 | 124 |
| 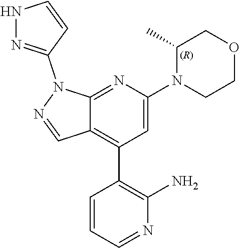 | 125 |
| 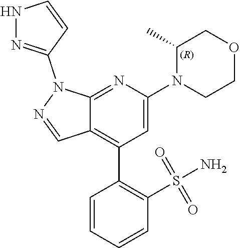 | 126 |
| 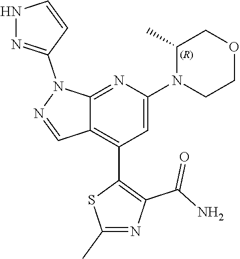 | 127 |
| 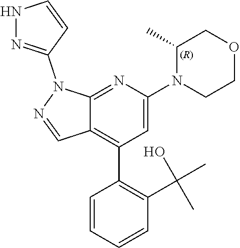 | 128 |
| 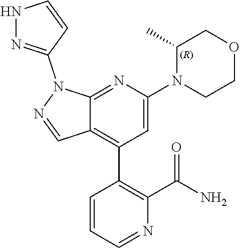 | 129 |
| 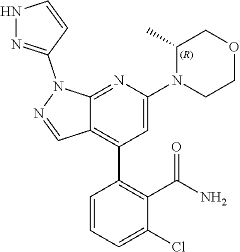 | 130 |
| 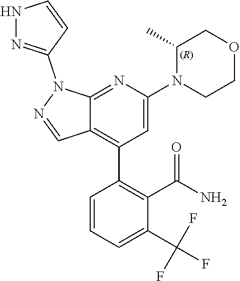 | 131 |
| 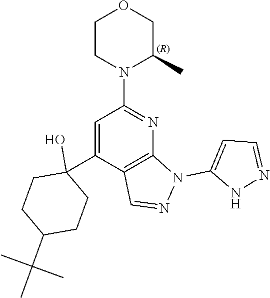 | 132 |
| 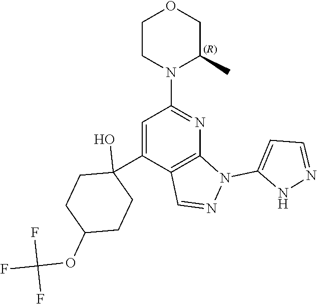 | 133 |
| 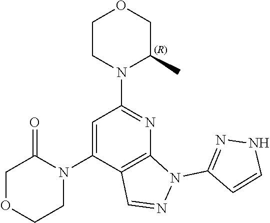 | 134 |
| 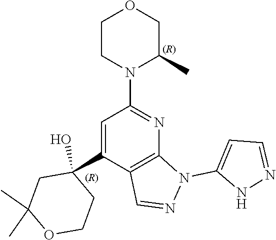 | 135 |
| 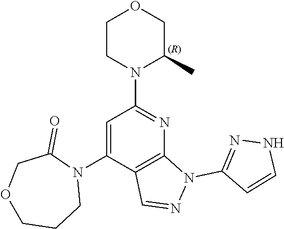 | 136 |
| 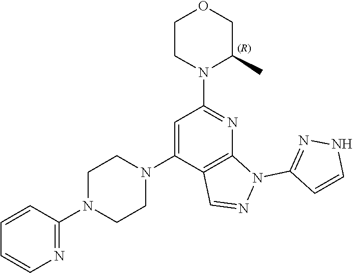 | 137 |
| 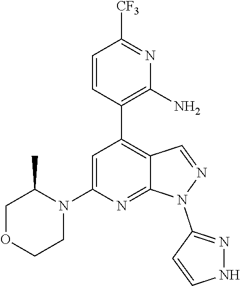 [SMILES: C[C@@H]1COCCN1c1cc(-c2ccc(C(F)(F)F)nc2N)c2cnn(-c3cc[nH]n3)c2n1] | 138 |
| 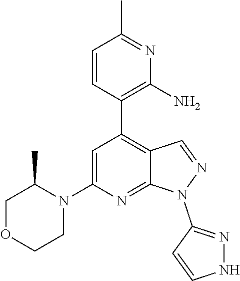 [SMILES: Cc1ccc(-c2cc(N3CCOC[C@H]3C)nc3c2cnn3-c2cc[nH]n2)c(N)n1] | 139 |
| 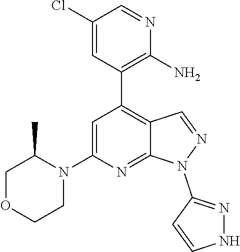 [SMILES: C[C@@H]1COCCN1c1cc(-c2cc(Cl)cnc2N)c2cnn(-c3cc[nH]n3)c2n1] | 140 |
| 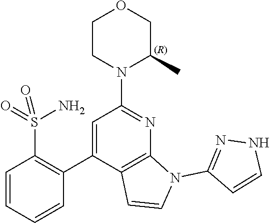 | 141 |
| 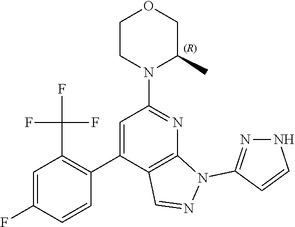 | 142 |
| 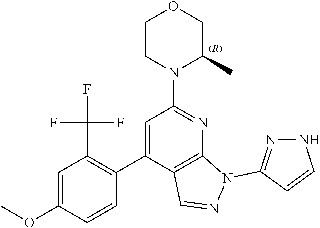 | 143 |
| 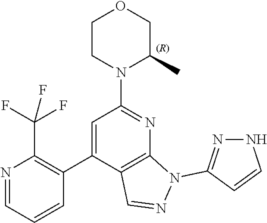 | 144 |
| 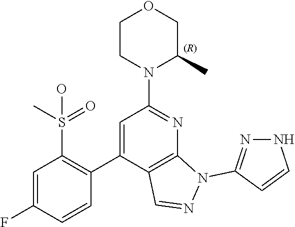 | 145 |
| 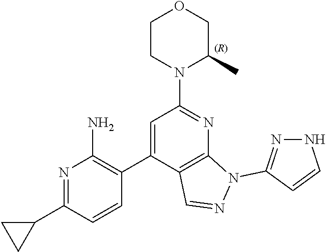 | 146 |
| 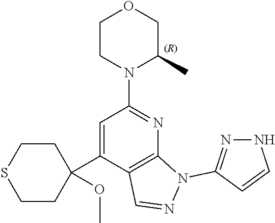 | 147 |
| 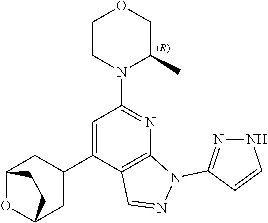 | 148 |
| 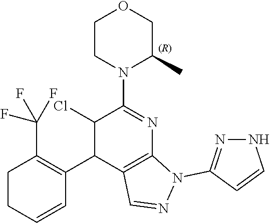 | 149 |
| 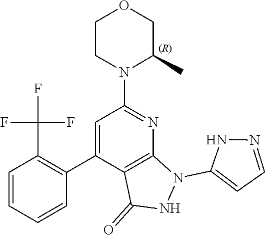 | 150 |
| 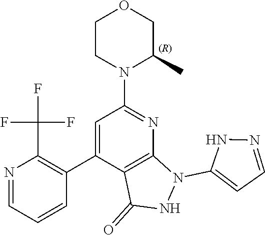 | 151 |
| 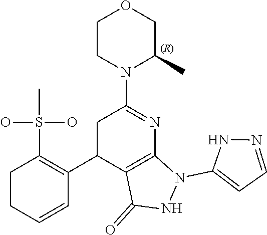 | 152 |

### Isomers and Compositions Thereof

The invention includes (where possible) individual diastereomers, enantiomers, epimers, and atropisomers of the compounds disclosed herein, and mixtures of diastereomers and/or enantiomers thereof including racemic mixtures. Although the specific stereochemistries disclosed herein are preferred, other stereoisomers, including diastereomers, enantiomers, epimers, atropisomers, and mixtures of these may also have utility in treating diseases. Inactive or less active diastereoisomers and enantiomers may be useful, e.g., for scientific studies relating to the receptor and the mechanism of activation.

It is understood that certain molecules can exist in multiple tautomeric forms. This invention includes all tautomers even though only one tautomer may be indicated in the examples.

The invention also includes pharmaceutically acceptable salts of the compounds, and pharmaceutical compositions including the compounds and a pharmaceutically acceptable carrier. The compounds are especially useful, e.g., in certain kinds of cancer and for slowing the progression of cancer once it has developed in a patient.

The compounds disclosed herein may be used in pharmaceutical compositions including (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound disclosed herein or a pharmaceutically acceptable salt thereof is the only active ingredient.

### Optical Isomers-Diastereomers-Geometric Isomers-Tautomers

Compounds disclosed herein may contain, e.g., one or more stereogenic centers and can occur as racemates, racemic mixtures, single enantiomers, individual diastereomers, and mixtures of diastereomers and/or enantiomers. The invention includes all such isomeric forms of the compounds disclosed herein. It is intended that all possible stereoisomers (e.g., enantiomers and/or diastereomers) in mixtures and as pure or partially purified compounds are included within the scope of this invention (i.e., all possible combinations of the stereogenic centers as pure compounds or in mixtures).

Some of the compounds described herein may contain bonds with hindered rotation such that two separate rotomers, or atropisomers, may be separated and found to have different biological activity which may be advantageous. It is intended that all of the possible atropisomers are included within the scope of this invention.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed by the invention.

Compounds disclosed herein having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

### Metabolites-Prodrugs

The invention includes therapeutically active metabolites, where the metabolites themselves fall within the scope of the claims. The invention also includes prodrugs, which are compounds that are converted to the claimed compounds as they are being administered to a patient or after they have been administered to a patient. The claimed chemical structures of this application in some cases may themselves be prodrugs.

### Isotopically Enriched Derivatives

The invention includes molecules which have been isotopically enriched at one or more position within the molecule. Thus, compounds enriched for deuterium fall within the scope of the claims.

### Methods of Preparing ATR Inhibitors

ATR inhibitors may be prepared using reactions and techniques known in the art. For example, certain ATR inhibitors may be prepared using techniques and methods disclosed in, e.g., International Application Nos. PCT/US2019/051539 and PCT/US2018/034729, each of which is incorporated by reference herein; U.S. Pat. Nos. 9,663,535, 9,549,932, 8,552,004, and 8,841,308, each of which is incorporated by reference herein; and U.S. Patent Application Publication No. 2019/0055240, which is incorporated by reference herein.

### Methods of Use

ATR inhibitors may be used for the treatment of a disease or condition having the symptom of cell hyperproliferation. For example, the invention described herein may be applicable for treatment of various oncological conditions harboring sensitizing gene mutations, such as tumors with any deleterious (loss-of-function) alterations in STAG2, SETD2, CDK12, ATRIP, REV3L, RAD17, CHTF8, FZR1, RAD51B, RAD51C, RAD51D, PALB2, RNASEH2A, or RNASEH2B. Mutations in this gene may be frequently found in the following tumor types: renal cell carcinoma, mature B-cell neoplasms, endometrial cancer, ovarian cancer, acute myeloid leukemia, colorectal cancer, skin cancer (non-melanoma), small bowel cancer, non-small cell lung cancer, melanoma, bladder cancer, pancreatic cancer, head and neck cancer, mesothelioma, glioma, prostate cancer, uterine cancer, breast cancer, stomach cancer and esophagogastric cancer. Accordingly, methods of the invention are preferably used in the treatment of these cancers.

In some embodiments, the loss of function is of ATRIP, and the cancer is uveal melanoma, cervical squamous cell carcinoma, endocervical adenocarcinoma, stomach adenocarcinoma, head and neck squamous cell carcinoma, or kidney renal clear cell carcinoma.

In some embodiments, the loss of function is of RNASEH2A or RNASEH2B, and the cancer is sarcoma, bladder urothelial carcinoma, chronic lymphocytic leukemia

In some embodiments, the loss of function is of CHTF8, and the cancer is prostate adenocarcinoma.

In some embodiments, the loss of function is of FZR1, and the cancer is head and neck squamous cell carcinoma, skin cutaneous melanoma, or kidney renal papillary cell carcinoma.

In some embodiments, the loss of function is of RAD17, and the cancer is stomach adenocarcinoma, prostate adenocarcinoma, or breast invasive carcinoma.

In some embodiments, the loss of function is of RAD51B, and the cancer is uterine corpus endometrial carcinoma, kidney renal papillary cell carcinoma, cervical squamous cell carcinoma, endocervical adenocarcinoma, skin cutaneous melanoma, or breast invasive carcinoma.

In some embodiments, the loss of function is of RAD51C, and the cancer is skin cutaneous melanoma, uterine corpus endometrial carcinoma, ovarian serous cystadenocarcinoma, or breast invasive carcinoma.

In some embodiments, the loss of function is of RAD51D, and the cancer is ovarian serous cystadenocarcinoma, stomach adenocarcinoma, or skin cutaneous melanoma.

In some embodiments, the loss of function is of REV3L, and the cancer is uterine corpus endometrial carcinoma, stomach adenocarcinoma, prostate adenocarcinoma, lung squamous cell carcinoma, cervical squamous cell carcinoma, endocervical adenocarcinoma, skin cutaneous melanoma, or lung adenocarcinoma.

In some embodiments, the loss of function is of SETD2, and the cancer is mesothelioma, kidney renal clear cell carcinoma, kidney renal papillary cell carcinoma, lung adenocarcinoma, cholangiocarcinoma, uveal melanoma, bladder urothelial carcinoma, cervical squamous cell carcinoma, endocervical adenocarcinoma, breast invasive carcinoma, lung squamous cell carcinoma, esophageal carcinoma, head and neck squamous cell carcinoma, or ovarian serous cystadenocarcinoma.

In some embodiments, the loss of function is of CDK12, and the cancer is prostate adenocarcinoma, ovarian serous cystadenocarcinoma, uterine corpus endometrial carcinoma, skin cutaneous melanoma, lung adenocarcinoma, or breast invasive carcinoma.

In some embodiments, the loss of function is of PALB2, and the cancer is prostate adenocarcinoma, lung adenocarcinoma, liver hepatocellular carcinoma, breast invasive carcinoma, uterine corpus endometrial carcinoma, lung squamous cell carcinoma, ovarian serous cystadenocarcinoma, or stomach adenocarcinoma.

Therapeutic methods of the invention include the step of administering a therapeutically effective amount of an ATR inhibitor to a subject in need thereof.

The disease or condition treated using methods of the invention may have the symptom of cell hyperproliferation. For example, the disease or condition may be a cancer. The cancer may be, e.g., carcinoma, sarcoma, adenocarcinoma, lymphoma, leukemia, or melanoma. The cancer may be, e.g., a solid tumor.

Non-limiting examples of cancers include prostate cancer, breast cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, pancreatic cancer, kidney cancer, bladder cancer, uterine cancer, colon cancer, and liver cancer.

Preferably, methods of the invention are used in the treatment of renal cell carcinoma, mature B-cell neoplasms, endometrial cancer, ovarian cancer, fallopian tube cancer, primary peritoneal cancer, colorectal cancer, skin cancer (non-melanoma), small bowel cancer, non-small cell lung cancer, melanoma, bladder cancer, pancreatic cancer, head and neck cancer, mesothelioma, glioma, prostate cancer, breast cancer, or esophagogastric cancer.

Non-limiting examples of carcinomas include medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Non-limiting examples of sarcomas include chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Non-limiting examples of leukemias include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

Non-limiting examples of melanomas include acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., 40 mesh.

### Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 0.05-1000 mg (e.g., 0.25-800 mg). In some embodiments, 0.05, 0.1, 0.25, 0.5, 1, 2, 3, 4, 5, 10, 15, 20, 25, 30, 35, 40, 45, 50, 100, 150, 200, 250, 300, 350, 400, 450, 500, 550, 600, 650, 700, 750, 800, 850, 900, 950, or 1000 mg of the compound is administered.

In the methods of the invention, the time period during which multiple doses of a compound of the invention are administered to a patient can vary. For example, in some embodiments, doses of the compounds of the invention are administered to a patient over a time period that is 1-7 days; 1-12 weeks; or 1-3 months. In other embodiments, the compounds are administered to the patient over a time period that is, for example, 4-11 months or 1-30 years. In other embodiments, the compounds are administered to a patient at the onset of symptoms. In any of these embodiments, the amount of compound that is administered may vary during the time period of administration. When a compound is administered daily, administration may occur, for example, 1, 2, or 3 times per day.

### Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to subjects in need thereof. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention. Oral administration is a preferred route of administration in the methods of the invention.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution- or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

The following examples are meant to illustrate the invention. They are not meant to limit the invention in any way.

### EXAMPLES

### Example 1. ATR Inhibition in STAG2-Deficient Cells

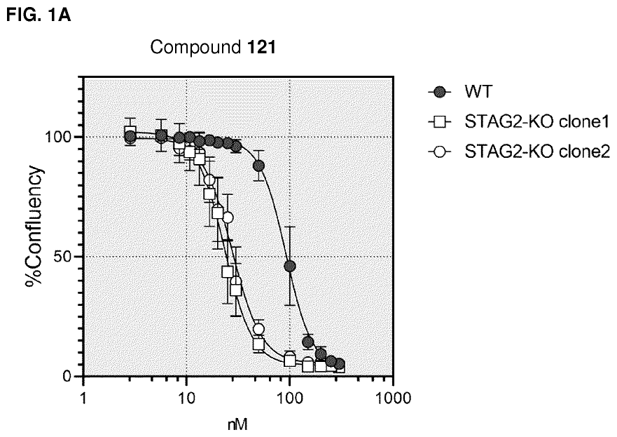

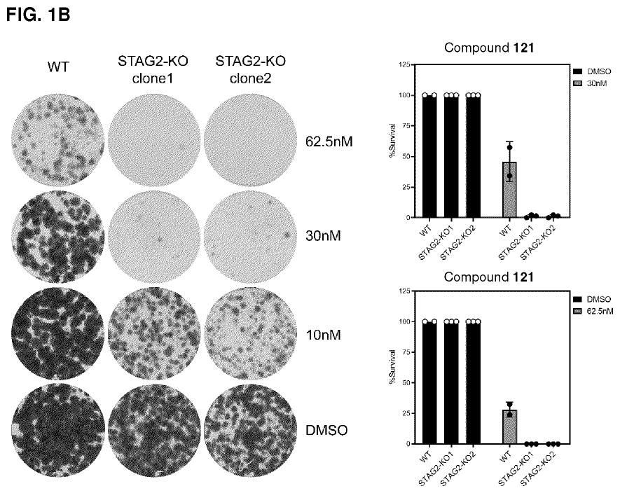

To assess the impact of STAG2 deficiency in the response to ATRi, we generated RPE1-hTERT-ΔTP53 deficient for STAG2 by knocking out the gene using CRISPR-Cas9. To avoid potential off-target effects, two distinct sgRNA were used to generate each KO clone. Treatment with compound 121 induced a significantly stronger inhibition of proliferation in STAG2-KO cells compare to their wild-type (WT) counterpart (FIG. 1A). This phenotype was observed in a second assay measuring viability through the ability of surviving cells to form colonies (FIG. 1B).

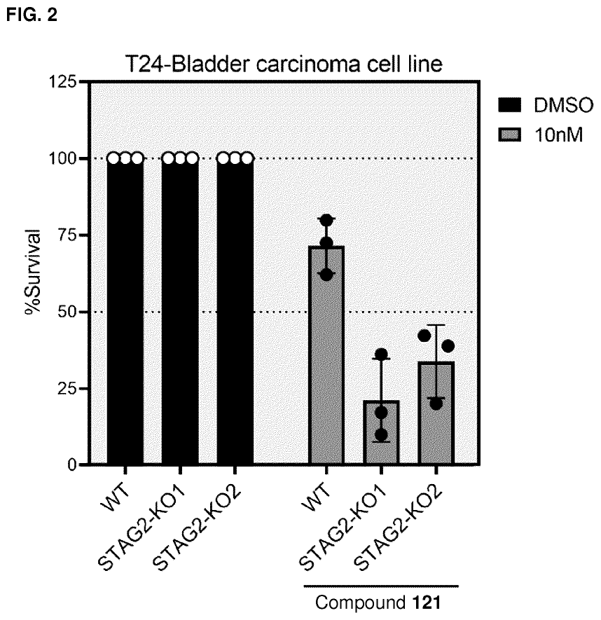

Because bladder carcinoma is a cancer where STAG2-deficient tumors have been observed, we sought to recapitulate our observation in a cell model originated from bladder carcinoma. Using a similar approach as previously, we generated T24 cells deficient for STAG2. Interestingly, colony formation of T24-STAG2 KO cells was impaired following compound 121 treatment (FIG. 2). Altogether, this demonstrates that STAG2 loss of function is a determinant of ATRi sensitivity. Those results suggest STAG2 as a potential biomarker for treatment of patients with ATRi.

### Example 2. ATR Inhibition in SETD2-Deficient Tumor Cells

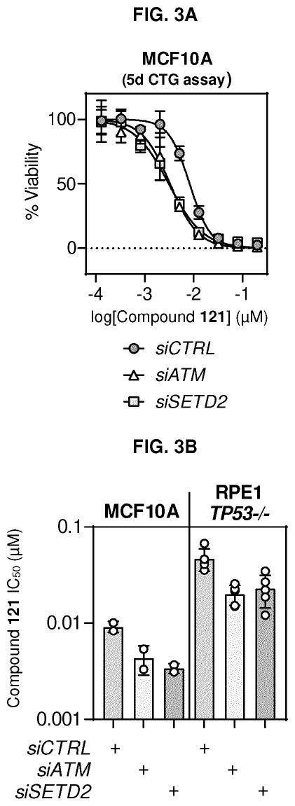

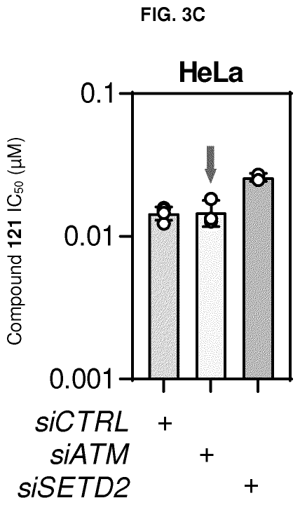

To determine whether SETD2 inactivation leads to ATRi sensitivity, we depleted SETD2 from two human cell lines, MCF10A and RPE1-hTERT-ΔTP53 using specific siRNAs. We observed that SETD2 depletion sensitized these cells to compound 121 treatment to a similar extent as depletion of ATM, a known determinant of sensitivity to ATRi (FIGS. 3A and 3B). Of note, depletion of SETD2 from a third cell line, HeLa, did not lead to ATRi sensitivity. However, in this cell line even depletion of ATM did not cause sensitivity to compound 121, suggesting that the HeLa cell line may not be a relevant model to study ATRi sensitivity with siRNA depletion (FIG. 3C).

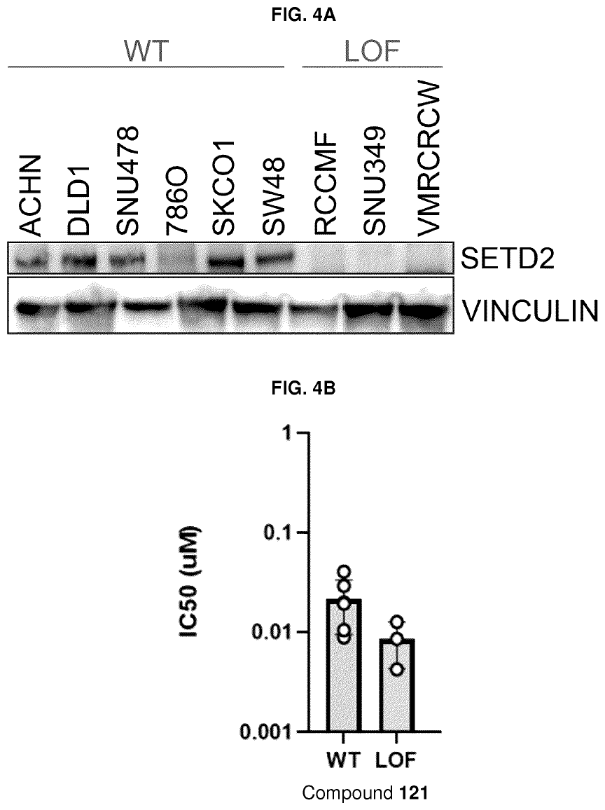

To study whether SETD2 deficiency leads to ATRi sensitivity in a more clinically relevant context, we assembled a panel of six SETD2-proficient and three SETD2-deficient renal clear cell carcinoma and colorectal carcinoma cell lines (FIG. 4A) and treated them with compound 121. We observed that, on average, the SETD2-deficient cell lines were more sensitive to compound 121 than SETD2-proficient ones (FIG. 4B).

Furthermore, the monotherapy dose escalation phase of the study (Module 1) enrolled patients with advanced solid tumors with eligible loss of function mutations in a predefined subset of genes associated with ATRi sensitivity (STEP2 genes). Multiple tumors and genomic alterations were represented including 5 patients with SETD2 mutations. Three of these patients remain on study with duration of treatment ranging from approximately 23 to 45 weeks. In addition, one of these patients has had a confirmed partial response (RECIST v1.1). As of the most recent reporting, the Clinical Benefit Rate (CBR) for the SETD2 patients evaluable for response was 80%. Meaning 4 out of 5 patients had either a response or were on therapy without progression ≥16 weeks. Recruitment of patients with tumors harboring SETD2 alterations is ongoing.

In conclusion, these data support SETD2 status as a biomarker of ATRi sensitivity in tumors.

### Example 3. ATR Inhibition in CDK12-Deficient Tumor Cells

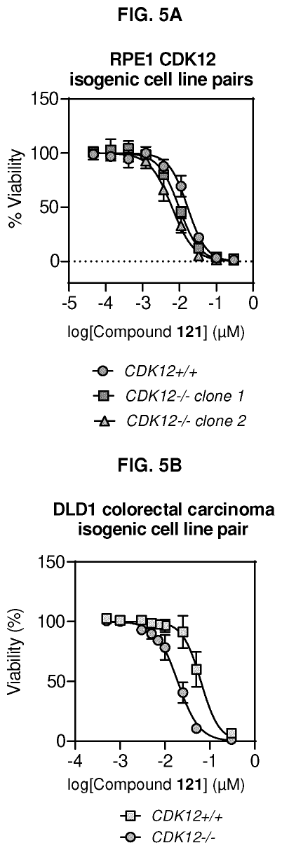

To assess the effect of CDK12 inactivation on ATRi sensitivity we used isogenic cell line pairs in the RPE1-hTERT-ΔTP53 and DLD1 human cell line backgrounds, where we compared clones carrying a CRISPR/Cas9-generated knockout of the CDK12 gene (CDK12−/−) with the parental, CDK12-wild type controls (CDK12+/+). We confirmed that in both cell line backgrounds, CDK12-deficiency led to increased sensitivity to compound 121 treatment (FIGS. 5A and 5B).

In an ongoing study, 7 CDK12 patients enrolled on M1 of the study were evaluable for response but none remain on study. One of these patients had a confirmed partial response (RECIST v1.1) and remained on study for close to 27 weeks. The Clinical Benefit Rate (CBR) for the CDK12 patients enrolled in Module 1 of the study was 29%, meaning 2 out of 7 patients had either a response or were on therapy without progression ≥16 weeks. Recruitment of patients with tumors harboring CDK12 alterations is ongoing. Combined, our data suggest that CDK12 status in tumors is a determinant of ATRi sensitivity.

### Example 4. ATR Inhibition in RNASEH2A- and RNASEH2B-Deficient Tumor Cells

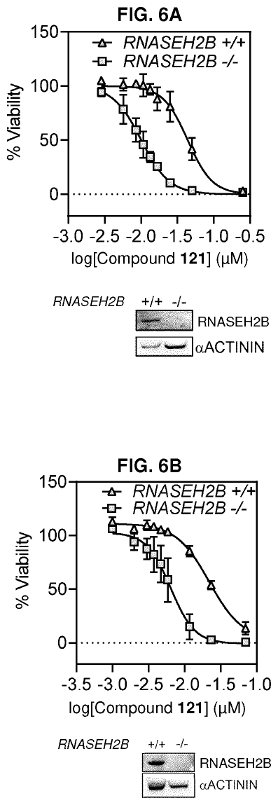

The RNase H2 protein complex is composed of three subunits encoded by the RNASEH2A, RNASEH2B and RNASEH2C genes in a manner, where all three subunits are required for the complex' stability and activity. To determine whether RNase H2 deficiency increases cellular sensitivity to ATRi, we inactivated the RNASEH2B gene in two human cell lines, RPE1-hTERT-ΔTP53 and 5637, using CRISPR/Cas9 (note that this leads to complete loss of the RNase H2 complex). In both cellular backgrounds, RNase H2-deficient cells were (RNASEH2B−/−) more sensitive to compound 121 as compared to isogenic wild-type (RNASEH2B+/+) cell lines (FIGS. 6A and 6B).

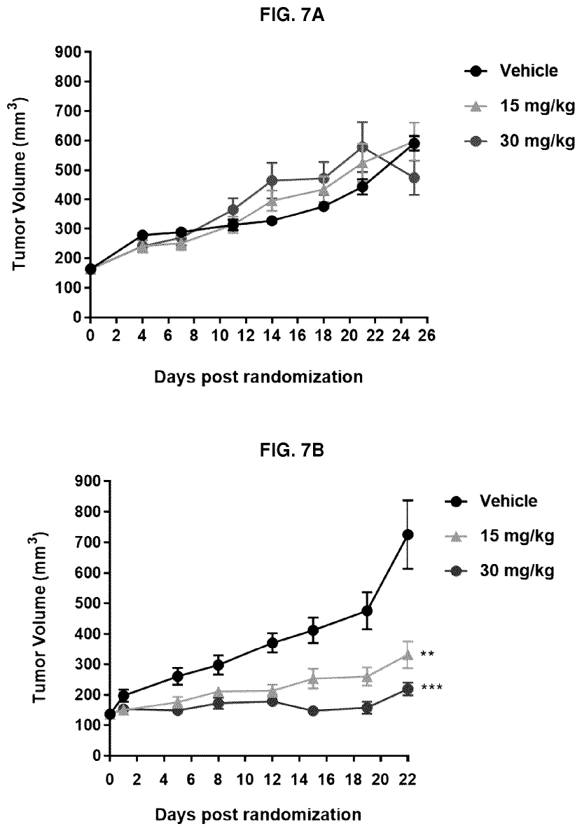

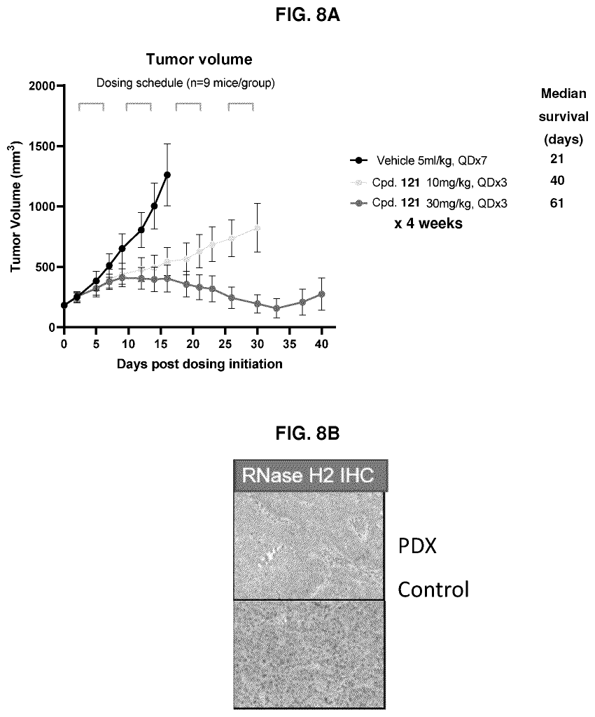

To confirm that RNase H2-deficiency sensitizes to ATRi treatment also in vivo, we developed the isogenic 5637 RNASEH2B+/+ and RNASEH2B−/− bladder carcinoma cell lines as subcutaneous xenografts in immunodeficient mice and treated them with compound 121 or a vehicle control. Whereas compound 121 showed no efficacy in RNASEH2B+/+ tumors, we observed profound and dose-dependent tumor growth inhibition in RNASEH2B−/− tumors upon compound 121 treatment (FIGS. 7A and 7B). Furthermore, we identified a patient-derived xenograft (PDX) of a triple negative breast cancer, that was RNase H2-deficient as confirmed by immunohistochemistry with a RNase H2-specific antibody. This model was also responsive to compound 121 treatment and showed tumor regressions at the maximum tolerated dose (FIGS. 8A and 8B).

Collectively, these data support using RNase H2 (RNASEH2B or RNASEH2A) alterations as a biomarker for ATRi sensitivity.

### Example 5. ATR Inhibition in RAD51B, RAD51C or RAD51D-Deficient Tumor Cells

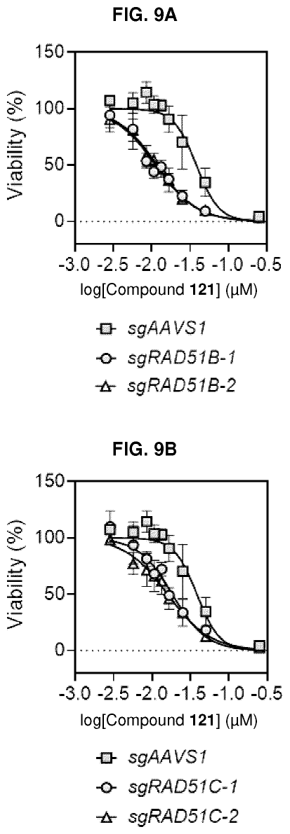

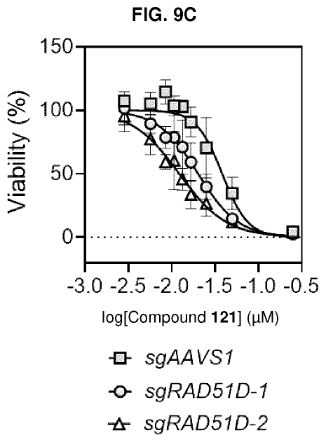

To determine the impact of RAD51B, RAD51C or RAD51D deficiency on ATRi sensitivity we individually inactivated these genes in Cas9-expressing RPE1-hTERT-ΔTP53 cells using two independent sgRNAs per gene. We observed that inactivation either of the three genes (RAD51B, RAD51C or RAD51D) sensitized cells to compound 121 treatment to a comparable extent (FIGS. 9A, 9B, 9C).

Furthermore, three patients with RAD51C alterations enrolled on M1 of the study were evaluable for response and all remain on study. One of these patients had a confirmed complete response (RECIST v1.1) and remains on study for 41 weeks. In addition, one patient had a CA-125 tumor marker response and remains on study for 43 weeks. The Clinical Benefit Rate (CBR) for the RAD51C patients enrolled in Module 1 of the study was 100%, meaning 3 out of 3 patients had either a response or are on therapy without progression ≥16 weeks. Recruitment of patients with tumors harboring RAD51C alterations is ongoing.

Altogether, these data support a conclusion that RAD51B, RAD51C, or RAD51D alterations are biomarkers of ATRi sensitivity.

### Example 6. ATR Inhibition in Cells Deficient for ATRIP, RAD17, REV3L, FZR1 or CHTF8

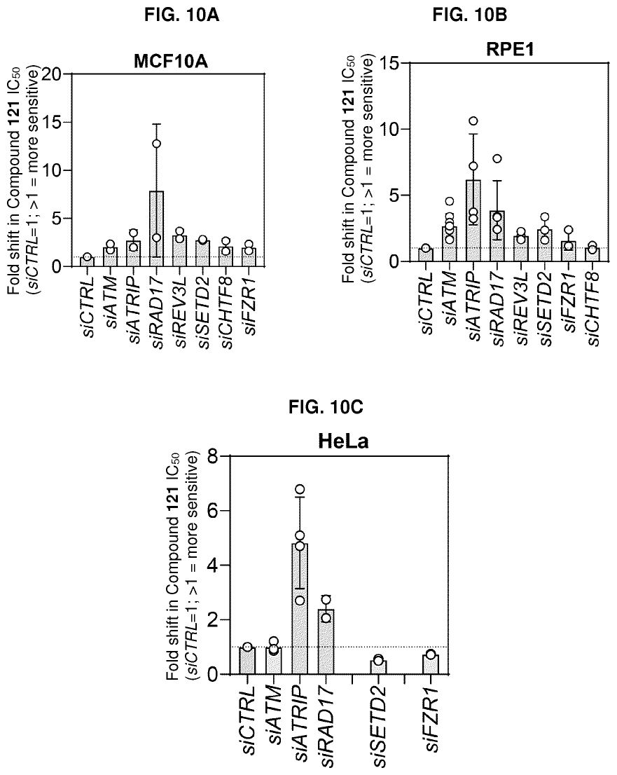

To investigate whether deficiency in any of the ATRIP, RAD17, REV3L, FZR1 or CHTF8 genes leads to ATRi sensitivity, we downregulated their expression in two or three human cell lines, RPE1-hTERT-ΔTP53, MCF10A or HeLa using specific siRNAs. siRNAs targeting SETD2 or ATM were used as positive controls. We confirmed that siRNA-mediated knockdown of ATRIP, RAD17, REV3L, FZR1 or CHTF8 sensitized at least one cell line to compound 121 treatment to a comparable, or greater level as compared to knockdown of ATM or SETD2 (FIGS. 10A, 10B, and 10C). These data suggest that tumors carrying inactivating alterations in ATRIP, RAD17, REV3L, FZR1 or CHTF8 may be sensitive to ATRi treatment.

### Example 7. Lack of ATRi Sensitivity with a ARID1A

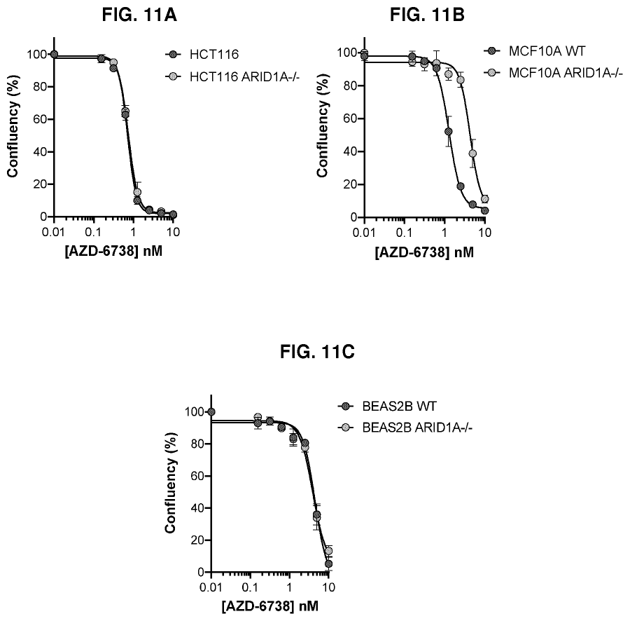

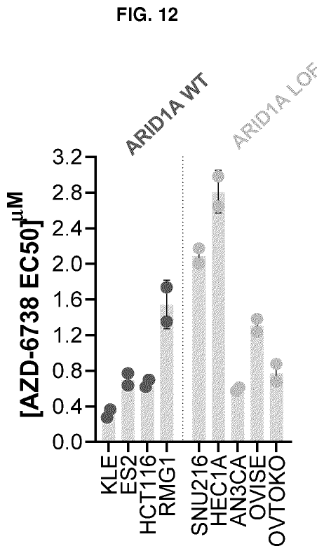

In addition to the genes disclosed herein, we investigated ATRi sensitivity to the loss of ARID1A, which was previously described in Williamson et al., Nat. Comms., 7:13837, 2016, as a bona fide biomarker of ATRi sensitivity. Three isogenic ARID1A wild-type (WT) and ARID1A-deficient (ARID1A−/−) human cell line pairs were employed: HCT116, MCF10A and BEAS2B. In all cases, ARID1A deficiency did not lead to sensitivity to the ATR inhibition by AZD-6738, contrary to published data (FIGS. 11A, 11B, and 11C). To strengthen this observation, we assembled a panel of tissue-matched cancer cell lines that were either ARID1A WT or carried deleterious ARID1A alterations (LOF) and measured their sensitivity to AZD-6738. Consistent with the isogenic cell line data, there was no significant trend towards higher ATRi sensitivity in ARID1A LOF cell lines as compared to ARID1A WT (FIG. 12). In aggregate, we believe that the genes named in this Application present a list of bona fide determinants of ATRi sensitivity as opposed to certain 5 biomarkers described in the scientific literature.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of treating a cancer in a subject, the method comprising administering to the subject in need thereof a therapeutically effective amount of an ATR inhibitor, wherein the cancer has been previously identified as a cancer having a loss of function of STAG2, SETD2, CDK12, ATRIP, REV3L, RAD17, CHTF8, FZR1, RAD51B, RAD51C, RAD51D, PALB2, RNASEH2A, or RNASEH2B.

2. A method of treating a cancer in a subject, the method comprising administering to the subject in need thereof a therapeutically effective amount of an ATR inhibitor, wherein the cancer has a loss of function of STAG2, SETD2, CDK12, ATRIP, REV3L, RAD17, CHTF8, FZR1, RAD51B, RAD51C, RAD51D, PALB2, RNASEH2A, or RNASEH2B.

3. A method of treating a cancer in a subject, the method comprising:
(i) identifying the cancer as having a loss of function of STAG2, SETD2, CDK12, ATRIP, REV3L, RAD17, CHTF8, FZR1, RAD51B, RAD51C, RAD51D, PALB2, RNASEH2A, or RNASEH2B; and
(ii) administering to the subject in need thereof a therapeutically effective amount of an ATR inhibitor.

4. The method of any one of claims 1 to 3, wherein the route of administration is an oral administration.

5. A method of inducing cell death in an aberrant cancer cell having a loss of function of STAG2, SETD2, CDK12, ATRIP, REV3L, RAD17, CHTF8, FZR1, RAD51B, RAD51C, RAD51D, PALB2, RNASEH2A, or RNASEH2B, the method comprising contacting the cell with an effective amount of an ATR inhibitor, the effective amounts being sufficient to induce cell death in the aberrant cancer cell.

6. A method of treating a cancer having a biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation in a subject, the method comprising administering to the subject in need thereof an effective amount of an ATR inhibitor.

7. A method of treating a cancer in a subject, the method comprising administering to the subject in need thereof an effective amount of an ATR inhibitor, wherein the cancer has been previously identified as having a biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation.

8. A method of inducing a cell death in a cancer cell having a STAG2, SETD2, CDK12, ATRIP, REV3L, RAD17, CHTF8, FZR1, RAD51B, RAD51C, RAD51D, PALB2, RNASEH2A, or RNASEH2B loss of function mutation, the method comprising contacting the cell with an ATR inhibitor.

9. The method of claim 8, wherein the cell is in a subject.

10. The method of any one of claims 6 to 9, wherein the method further comprises identifying the cancer as having a biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation prior to the administering step or contacting step.

11. The method of claim 10, wherein the identifying step comprises:
from read counts for a plurality of single nucleotide variants (SNVs) comprising homozygous and heterozygous SNVs obtained from sequencing a sample comprising the cancer cell and from reference read counts, determining an integer total copy number of a locus segment within a STAG2, SETD2, CDK12, ATRIP, REV3L, RAD17, CHTF8, FZR1, RAD51B, RAD51C, RAD51D, PALB2, RNASEH2A, or RNASEH2B gene region in a cancer cell from the subject or in the cancer cell and/or two integer allele-specific copy numbers of the locus segment,
wherein the cancer is identified as having a biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation,
if at least one of the integer total copy numbers and the integer allele-specific copy numbers is 0, provided that the remaining STAG2, SETD2, CDK12, ATRIP, REV3L, RAD17, CHTF8, FZR1, RAD51B, RAD51C, RAD51D, PALB2, RNASEH2A, or RNASEH2B allele, if present, comprises an inactivating mutation, or
if none of the integer allele-specific copy numbers is 0 and STAG2, SETD2, CDK12, ATRIP, REV3L, RAD17, CHTF8, FZR1, RAD51B, RAD51C, RAD51D, PALB2, RNASEH2A, or RNASEH2B alleles are present, each of the alleles independently comprises an inactivating mutation.

12. The method of claim 11, wherein the determining step comprises:
from the read counts and the reference read counts, determining total copy number log-ratios, allelic copy number log-odds ratios, and target coverage values for the SNVs;
segmenting the total copy number log-ratios and the allelic copy number log-odds ratios;
estimating sample purity and sample ploidy for the cancer cell from the total copy number log-ratios and the target coverage values; and
from the target coverage values, the sample purity, the sample ploidy, the total copy number log-ratios, and the allelic copy number log-odds ratios, generating an integer total copy number of a segment comprising a plurality of SNVs within a STAG2, SETD2, CDK12, ATRIP, REV3L, RAD17, CHTF8, FZR1, RAD51B, RAD51C, RAD51D, PALB2, RNASEH2A, or RNASEH2B gene region in the cancer cell and two integer allele-specific copy numbers of the segment.

13. The method of claim 12, wherein the method further comprises adjusting the ratios for location shift.

14. The method of any one of claims 11 to 13, wherein the plurality of SNVs comprises consistently covered SNVs.

15. The method of claim 14, wherein each of the consistently covered SNVs has the mean coverage of at least 200× reads across panel of normal samples.

16. The method of any one of claims 11 to 15, wherein the plurality of SNVs comprises frequent SNVs, the frequent SNVs having an allele frequency of 33% to 66% in humans.

17. The method of claim 16, wherein the plurality of SNVs comprises SNVs proximal to the frequent SNVs.

18. The method of any one of claims 11 to 16, wherein the plurality of SNVs comprises SNVs, each of the SNVs having a 5′-flanking sequence of at least 20 contiguous nucleobases comprising 25-75% GC content, wherein the 5′-flanking sequence is unique and does not comprise other SNVs.

19. The method of any one of claims 11 to 18, wherein the plurality of SNVs comprises at least 20 heterozygous SNVs.

20. The method of any one of claims 11 to 19, wherein the reference read counts are from a panel of normal samples.

21. The method of any one of claims 11 to 19, wherein the gene region comprises flanking regions up to 10 kilobases each.

22. The method of any one of claims 11 to 19, wherein the gene region comprises flanking regions up to 5 kilobases each.

23. The method of any one of claims 11 to 19, wherein the gene region comprises flanking regions up to 2 kilobases each.

24. The method of any one of claims 11 to 23, wherein the gene region is an exome region.

25. The method of any one of claims 11 to 23, wherein the gene region is a transcriptome region.

26. The method of any one of claims 11 to 23, wherein the gene region is a genome region.

27. The method of any one of claims 6 to 26, wherein the biallelic loss of function mutation comprises at least one somatic mutation.

28. The method of any one of claims 6 to 27, wherein the biallelic loss of function mutation comprises at least one germline mutation.

29. The method of any one of claims 1 to 28, wherein the ATR inhibitor is a compound of formula (I): 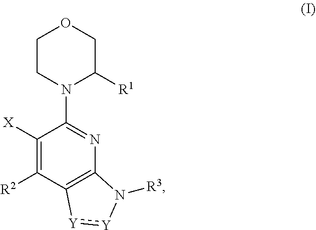
or a pharmaceutically acceptable salt thereof,
wherein= [symbol]
is a double bond, and each Y is independently N or CR⁴; or [symbol] is a single bond, and each Y is independently NR^{Y}, carbonyl, or C(R^{Y})₂; wherein each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

30. The method of claim 29, wherein the ATR inhibitor is a compound of formula (II): 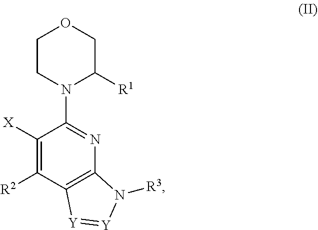
or a pharmaceutically acceptable salt thereof,
wherein
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

31. The method of claim 29, wherein the ATR inhibitor is selected from the group consisting of compounds 43, 57, 62, 87, 93, 94, 95, 99, 100, 106, 107, 108, 109, 111, 112, 113, 114, 115, 116, 118, 119, 120, 121, 122, 123, 135, 147, 148, and pharmaceutically acceptable salts thereof.

32. The method of claim 31, wherein the ATR inhibitor is compound 43 or a pharmaceutically acceptable salt thereof.

33. The method of claim 29, wherein the ATR inhibitor is compound 121 or a pharmaceutically acceptable salt thereof.

34. The method of claim 29, wherein the ATR inhibitor is compound 122 or a pharmaceutically acceptable salt thereof.

35. The method of any one of claims 1 to 28, wherein the ATR inhibitor is 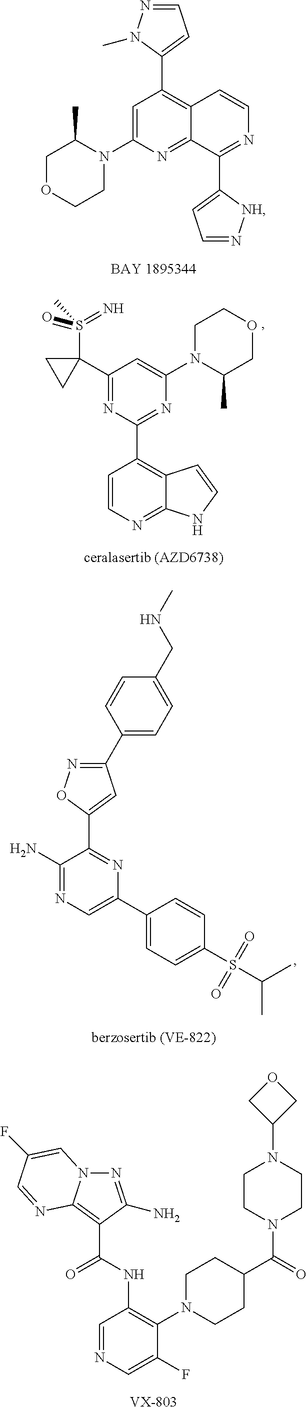
or a pharmaceutically acceptable salt thereof.

36. The method of any one of claims 1 to 28, wherein the ATR inhibitor is 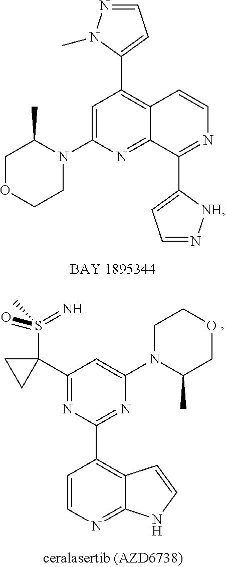 [SMILES: C[C@@H]1COCCN1c1cc(-c2ccnn2C)c2ccnc(-c3ccn[nH]3)c2n1 ; C[C@@H]1COCCN1c1cc(C2([S@](C)(=N)=O)CC2)nc(-c2ccnc3[nH]ccc23)n1]
or a pharmaceutically acceptable salt thereof.

37. The method of any one of claims 1 to 36, wherein the method does not comprise the step of administering or contacting with a PARP inhibitor.

38. The method of any one of claims 1 to 37, wherein the ATR inhibitor is administered as a monotherapy.

39. The method of any one of claims 1 to 36, wherein the cancer is renal cell carcinoma, mature B-cell neoplasm, endometrial cancer, ovarian cancer, fallopian tube cancer, primary peritoneal cancer, colorectal cancer, skin cancer, small bowel cancer, non-small cell lung cancer, melanoma, bladder cancer, pancreatic cancer, head and neck cancer, mesothelioma, glioma, prostate cancer, breast cancer, or esophagogastric cancer.

40. The method of any one of claims 1 to 36, wherein the loss of function is a loss of function of STAG2.

41. The method of claim 37, wherein the cancer is renal cell carcinoma, endometrial cancer, acute myeloid leukemia, bladder cancer, uterine cancer, or stomach cancer.

42. The method of any one of claims 1 to 36, wherein the loss of function is a loss of function of SETD2.

43. The method of claim 42, wherein the cancer is mesothelioma, kidney renal clear cell carcinoma, kidney renal papillary cell carcinoma, lung adenocarcinoma, cholangiocarcinoma, uveal melanoma, bladder urothelial carcinoma, cervical squamous cell carcinoma, endocervical adenocarcinoma, breast invasive carcinoma, lung squamous cell carcinoma, esophageal carcinoma, head and neck squamous cell carcinoma, or ovarian serous cystadenocarcinoma.

44. The method of any one of claims 1 to 36, wherein the loss of function is a loss of function of CDK12.

45. The method of claim 44, wherein the cancer is prostate adenocarcinoma, ovarian serous cystadenocarcinoma, uterine corpus endometrial carcinoma, skin cutaneous melanoma, lung adenocarcinoma, or breast invasive carcinoma.

46. The method of any one of claims 1 to 36, wherein the loss of function is a loss of function of ATRIP.

47. The method of claim 46, wherein the cancer is uveal melanoma, cervical squamous cell carcinoma, endocervical adenocarcinoma, stomach adenocarcinoma, head and neck squamous cell carcinoma, or kidney renal clear cell carcinoma.

48. The method of any one of claims 1 to 36, wherein the loss of function is a loss of function of REV3L.

49. The method of claim 48, wherein the cancer is uterine corpus endometrial carcinoma, stomach adenocarcinoma, prostate adenocarcinoma, lung squamous cell carcinoma, cervical squamous cell carcinoma, endocervical adenocarcinoma, skin cutaneous melanoma, or lung adenocarcinoma.

50. The method of any one of claims 1 to 36, wherein the loss of function is a loss of function of RAD17.

51. The method of claim 50, wherein the cancer is stomach adenocarcinoma, prostate adenocarcinoma, or breast invasive carcinoma.

52. The method of any one of claims 1 to 36, wherein the loss of function is a loss of function of CHTF8.

53. The method of claim 52, wherein the cancer is prostate adenocarcinoma.

54. The method of any one of claims 1 to 36, wherein the loss of function is a loss of function of FZR1.

55. The method of claim 54, wherein the cancer is head and neck squamous cell carcinoma, skin cutaneous melanoma, or kidney renal papillary cell carcinoma.

56. The method of any one of claims 1 to 55, wherein the loss of function is a loss of function of RAD51B.

57. The method of claim 56, wherein the cancer is uterine corpus endometrial carcinoma, kidney renal papillary cell carcinoma, cervical squamous cell carcinoma, endocervical adenocarcinoma, skin cutaneous melanoma, or breast invasive carcinoma.

58. The method of any one of claims 1 to 36, wherein the loss of function is a loss of function of RAD51C.

59. The method of claim 58, wherein the cancer is skin cutaneous melanoma, uterine corpus endometrial carcinoma, ovarian serous cystadenocarcinoma, or breast invasive carcinoma.

60. The method of any one of claims 1 to 36, wherein the loss of function is a loss of function of RAD51D.

61. The method of claim 60, wherein the cancer is ovarian serous cystadenocarcinoma, stomach adenocarcinoma, or skin cutaneous melanoma.

62. The method of any one of claims 1 to 36, wherein the loss of function is a loss of function of PALB2.

63. The method of claim 62, wherein the cancer is prostate adenocarcinoma, lung adenocarcinoma, liver hepatocellular carcinoma, breast invasive carcinoma, uterine corpus endometrial carcinoma, lung squamous cell carcinoma, ovarian serous cystadenocarcinoma, or stomach adenocarcinoma.

64. The method of any one of claims 1 to 36, wherein the loss of function is a loss of function of RNASEH2A.

65. The method of any one of claims 1 to 36, wherein the loss of function is a loss of function of RNASEH2B.

66. The method of claim 64 or 65, wherein the cancer is sarcoma, bladder urothelial carcinoma, chronic lymphocytic leukemia.
